(19) United States
(12) Patent Application Publication
(10) Pub. No.: US 2004/0068187 A1
(43) Pub. Date: Apr. 8, 2004

(54) Computer-aided orthopedic surgery

(76) Inventors: Norman M. Krause, Pittsburgh, PA (US); Lee E. Weiss, Pittsburgh, PA (US); Kenji Shimada, Pittsburgh, PA (US); Takeo Kanade, Pittsburgh, PA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/636,311

(22) Filed: Aug. 7, 2003

Related U.S. Application Data
(63) Continuation-in-part of application No. 09/545,685, filed on Apr. 7, 2000.
(63) Continuation of application No. 09/694,665, filed on Oct. 23, 2000.

(51) Int. Cl. A61B 8/00

(52) U.S. Cl. 600/443; 128/916

(57) ABSTRACT

Systems and method for generating three dimensional (3D) models of bones are described. In one embodiment, a method of generating a 3D model of a bone can include determining contours of the bone based two dimensional (2D) images of the bone and, based on the contours, modifying a 3D template model of the bone to generate a 3D model of the bone.

### CROSS REFERENCE TO RELATED APPLICATIONS

This application is a continuation-in-part of U.S. patent application Ser. No. 09/545,685 filed on Apr. 7, 2000 and a continuation of U.S. patent application Ser. No. 09/694,665 filed on Oct. 23, 2000, the contents of both of which applications are expressly incorporated by reference herein in their entireties.

### BACKGROUND

1. Field

The present invention broadly relates to the field of orthopedic surgery, and more particularly, to computer assisted orthopedic surgery that uses two or more two dimensional images of a patient's bone to generate a computer-based 3D (three dimensional) model of the patient's bone and a computer-based surgical plan for the doctor.

The present invention generally relates to devices and methods for implementing computer-aided surgical procedures and more specifically relates to devices and methods for implementing a computer-aided orthopedic surgery utilizing intra-operative feedback.

2. Description of the Related Art

Bone distraction in orthopedic surgery might well be considered one of the earliest successful forms of tissue engineering. Bone distraction is a therapeutic process invented in Russia in about 1951 for treating fractures, lengthening limbs and correcting other skeletal defects such as angular deformities. In bone distraction, external fixators are used to correct bone deformities and to lengthen bones by the controlled application of ‘tension-stress’, resulting in natural, healthy tissue.

FIG. 1 illustrates a prior art Ilizarov fixator 20 attached to a bone 22. The external Ilizarov fixator 20 is constituted of a pair of rings 24 separated by adjustable struts 28. The rings 24 are mounted onto the bone 22 from outside of the patient's body through wires or half-pins 26 as illustrated in FIG. 1. The lengths of the struts 28 can be adjusted to control the relative positions and orientations of the rings 24. After the fixator 20 is mounted to the patient's bone 22, the bone 22 is cut by osteotomy (i.e., surgical cutting of a bone) as part of the bone distraction process. Thereafter, the length of each individual strut 28 is adjusted according to a surgical plan. This length adjustment results in the changing of the relative position of the rings 24, which then forces the distracted (or “cut”) bone ends to comply and produce new bone in-between. This is termed the principle of “tension-stress” as applied to bone distraction.

The bone distraction rate is usually controlled at approximately 1 mm (millimeter) per day. The new bone grows with the applied distraction and consolidates after the distraction is terminated. Thereafter, the fixator 20 can be safely removed from the bone 22 and, after recanalization, the new or “distracted” bone is almost indistinguishable from the old or pre-surgery bone. The bone 22 may be equipped with other units, such as hinges, to correct rotational deformities about one or a few fixed axes. Thus, controlled application of mechanical stress forces the regeneration of the bone and soft tissues to correct their own deformities. The whole process of deformity correction is known as “bone distraction.”

At present, the following nominal steps are performed during the bone distraction process: (1) Determine an appropriate frame size for the fixator (e.g., for the Ilizarov fixator 20); (2) Measure (e.g., from X-rays) the deformity of bone fragments (or the anticipated fragments after surgically cutting the bone) and obtain six parameters that localize one fragment relative to the other; (3) Determine (or anticipate) how the fixator frame should be mounted on the limb; (4) Input the parameters and measurements to a computer program that generates the strut lengths as a function of time required to correct the deformity; (5) Mount the fixator frame onto the bone fragments; and (6) Adjust the strut lengths on a daily basis according to the schedule generated in step (4).

The steps outlined in the preceding paragraph are currently executed with minimal computerized assistance. Typically, surgeons manually gather or determine the required data (e.g., fixator frame size, bone dimensions, fixator frame mounting location and orientation, etc.) and make their decisions based on hand-drawn two-dimensional sketches or using digitized drawings obtained by tracing X-ray images. For example, a computerized deformity analysis (CDA) and pre-operative planning system (hereafter “the CDA system”) developed by Orthographics of Salt Lake City, Utah, USA, creates the boundary geometry of bones using X-ray images that are first digitized manually, i.e., by placing an X-ray image on a light table and then tracing the outline with a digitizing stylus, and then the digital data are fed into the CDA system. Thereafter, the CDA system assists the surgeon in measuring the degree of deformity and to make a surgical plan. The entire process, however, is based on two-dimensional drawings and there is no teaching of showing or utilizing three-dimensional bone deformity or bone geometry.

It is observed that in the complex area of bone distraction surgery, it is difficult, if not impossible, to make accurate surgical plans based solely on a limited number of two-dimensional renderings of bone geometry. This is because of the complex and inherently three-dimensional nature of bone deformities as well as of fixator geometry. Furthermore, two-dimensional depictions of surgical plans may not accurately portray the complexities involved in accessing the target positions of the osteotomy and fixator pins surrounding the operated bone. Lack of three-dimensional modeling of these geometric complexities makes it difficult to accurately mount the fixator on the patient according to the pre-surgical plan.

After a surgeon collects the requisite data (e.g., fixator frame size to be used, patient's bone dimensions, fixator frame mounting location and orientation, etc.), the surgeon may use the simulation software accompanying commercially available fixators (such as the Taylor Spatial Frame distributed by Smith & Nephew Inc. of 1450 Brooks Road, Memphis, Tenn., USA 38116) to generate a day-by-day plan that shows how the lengths of the fixator struts should be adjusted. Such a plan is generated after the initial and target frame positions and orientations are specified by the surgeon. However, the only functionality of the simulation software is a simple calculation of the interpolated frame configurations. The software does not provide any assistance to the surgeon about making surgical plans nor does it provide any visual feedback on how the fixator frame and bone fragments should be moved over time.

The Taylor Spatial Frame (shown, for example, in FIG. 16) with six degrees of freedom (DOF) is more versatile, flexible and complex than the Ilizarov fixator 20 in FIG. 1. Because of the sophistication of modern fixators (e.g., the Taylor Spatial Frame) and because of the limitations of the presently available bone distraction planning and execution systems, current computerized bone distraction procedures are error-prone, even when performed by the most experienced surgeons. As a result, the patients must typically revisit the surgeon several times after the initial operation in order for the surgeon to re-plan and refine the tension-stress schedule, or even to re-position the fixator. Such reiterations of surgical procedures are not only time-consuming, but incur additional costs and may lead to poorer therapeutic results while unnecessarily subjecting patients to added distress. It is therefore desirable to generate requisite bone and fixator models in three-dimensions prior to surgery so as to minimize the surgery planning and execution errors mentioned hereinbefore.

The discussion given hereinbelow describes some additional software packages that are available today to assist in the simulation and planning of bone distraction. However, it is noted at the outset that these software packages are not based on three-dimensional models. Further, these software packages are quite limited in their capabilities to assist the surgeon in making important clinical and procedural decisions, such as how to access the site of the osteotomy or how to optimally configure fixator pin configurations. Additional limitations of the present software systems include: (1) No realistic three-dimensional view of a bone and a fixator; (2) No usage of animation in surgical simulation; (3) Lack of an easy-to-use graphical user interface for user-friendliness; (4) No on-line database of standard or past similar cases and treatment data; and (5) No file input/output to store or retrieve previous case data.

In “Correction of General Deformity With The Taylor Spatial Frame Fixator” (1997), Charles J. Taylor refers to a software package from Smith & Nephew (Memphis, Tenn.) (hereafter “the Smith software”) that utilizes the Taylor Spatial Frame for certain computations. However, the Smith software does not include any visual output to the user (i.e., the surgeon) and the user needs to enter all data via a dialog box. Being mechanical in nature, the strut locations in a fixator are static. However, the Smith software does not account for whether a strut can be set to all the lengths necessary during the bone correction process. Further, the Smith software cannot calculate corrections that are due to malrotation (of the fixator) only.

As described hereinbefore, a software for computerized bone deformity analysis and preoperative planning is developed by Orthographics of Salt Lake City, Utah, USA (hereafter “the Orthographics software”). The Orthographics software creates the boundary geometry of bones using X-ray images that are first digitized manually as previously mentioned. Thereafter, the Orthographics software assists the surgeon in measuring the degree of bone deformity and to make a surgical plan. The entire process, however, is based on two-dimensional drawings and there is no support for showing or utilizing three-dimensional bone deformity or bone geometry. However, it is difficult to make accurate surgical plans based on a few such two-dimensional renderings considering the complex, three-dimensional nature of bone deformities and fixator geometry, and also considering the complexity involved in accessing the target positions of the osteotomy and fixator pins. This inherently three-dimensional nature of bone geometry and fixator assembly also makes it difficult to accurately mount the fixator on the patient's bone according to the two-dimensional pre-surgical plan. For further reference, see D. Paley, H. F. Kovelman and J. E. Herzenberg, Ilizarov Technology, “Advances in Operative Orthopaedics,” Volume 1, Mosby Year Book, Inc., 1993.

The software developed by Texas Scottish Rite Hospital for Children utilizes primitive digitization of the radiographs to generate three-dimensional representations of bones without any simulation. Additionally, the generated models are very primitive and do not show any kind of detail on the bone. For further reference, see Hong Lin, John G. Birch, Mikhail L. Samchukov and Richard B. Ashman, “Computer Assisted Surgery Planning For Lower Extremity Deformity Correction By The Ilizarov Method,” Texas Scottish Rite Hospital for Children.

The SERF (Simulation Environment of a Robotic Fixator) software has capability to represent a three-dimensional bone model. However, the graphical representations of the fixator frame and the bone by the SERF software are over-simplified. Furthermore, there is no mention of any user interface except for a dialog box that prompts a user (e.g., a surgeon) for a “maximum distance.” Additional information may be obtained from M. Viceconti, A. Sudanese, A. Toni and A. Giunti, “A software simulation of tibial fracture reduction with external fixatoi,” Laboratory for Biomaterials Technology, Istituto Rizzoli, Bologna, Italy, and Orthopaedic Clinic, University of Bologna, Italy, 1993.

In “Computer-assisted preoperative planning (CAPP) in orthopaedic surgery,” Orthopaedic Hospital, Medical College, University of Zagreb, Yugoslavia, 1990, Vilijam Zdravkovic and Ranko Bilic describe a CAPP and Computer Assisted Orthopedic Surgery system. The system receives feedback and derives a bone's geometry from two two-dimensional scans. However, this system still uses the less sophisticated and less complex Ilizarov fixator 20 (FIG. 1) instead of the more advanced Taylor Spatial Frame.

In a computer-assisted surgery, the general goal is to allow the surgeon to accurately execute the pre-operative plan or schedule. One approach to fulfill this goal is to provide feedback to the surgeon on the relative positions and the orientations of bone fragments, fixator frame and osteotomy/coricotomy site as the surgical procedure progresses. These positions could be determined in real time by measuring, with the help of an infrared (IR) tracking system, the positions of infrared light emitting diode (LED) markers strategically placed on the fixator frame, on cutting tools and on the patient. The relative positions of all these objects (and deviations from the planned positions) could then be displayed via a computerized image simulation to give guidance to the surgeon operating on the patient. Such a feedback approach is currently used to help register acetabular implants in artificial hip surgery using an Optotrak optical tracking camera from Northern Digital Inc. of Ontario, Canada. The Optotrak camera is capable of tracking the positions of special LEDs or targets attached to bones, surgical tools and other pieces of operating room equipment. However, for use in a computer-aided bone distraction system, the Optotrak camera and additional display hardware are too expensive to consider for a widespread bone distraction commercialization strategy.

It is estimated that, at present, less than 1% of orthopedic surgeons practice the bone distraction procedure and less than 5000 bone distraction cases are performed per year worldwide. Such relative lack of popularity may be attributed to the fact that learning the techniques for bone distraction is extremely demanding and time-consuming. Therefore, the average orthopedic surgeon does not perform these techniques. Thus, there is a significant number of patients for whom external fixation with distraction would be the treatment of choice, but because of the current complexity and cost limitations, these patients never benefit from advanced bone distraction procedures.

It is therefore desirable to develop a user-friendly (i.e., a surgeon-friendly) system that would make bone distraction a viable option for a much broader market of surgeons than are currently using this therapy. It is also desirable to devise a computer-based surgical planning service that simplifies frame fixation, decreases preoperative planning time and reduces the chances of complications, thereby making frame fixation a relatively physician-friendly technique. To facilitate acceptance of complex bone distraction procedures to a wider segment of orthopedic surgeons, it is further desirable to overcome two primary limitations present in current surgical planning and execution software: (1) the lack of three-dimensional visual aids and user-friendly simulation tools, and (2) the lack of an accurate and economical registration (i.e., fixator mounting) scheme.

Poorly aligned or misaligned bones can occur for a variety of reasons including congenital deformity and/or accidental disfigurement. A bone can be characterized as having an actual (or anatomical) axis that runs through the cross-sectional center of the bone and a mechanical axis, that extends between the joints at either end of the bone and defines the movement of the bone. In a generally straight bone with joints in line with the anatomical axis, e.g., the tibia with the knee and ankle joints, the anatomical and the mechanical axes should almost coincide. In a nonlinear bone, e.g., the femur with off center hip joint, the mechanical axis and the anatomical axis do not coincide even when the bone is correctly aligned.

The essence of a bone deformity or disfigurement occurs when the anatomical axis is altered to a point that the mechanical (motion) axis is not in its desired position. In a straight bone such as the tibia, the amount of disfigurement can be calculated as the deviation between the anatomical axis and the mechanical axis (because the axes should align in a straight bone). This deviation can cause discomfort, join disease, decreased range of motion, and/or numerous other medical problems. To correct or limit these improper alignments, an orthopedic surgeon may perform corrective surgery on the deformed or disfigured bone to return symmetry between the axes.

One type of corrective orthopedic surgery is an osteotomy. Osteotomies are characterized by cutting one or more slices into a deformed bone to a depth sufficient to allow the bone to be “repositioned” in a way that aligns the actual axis of motion with the desired axis. Typically, the bone repositioning forms a “wedge” or gap of open space in the bone. This space is filled via bone graft to promote new bone growth, and some type of fixation mechanism is attached to the bone to keep the bone in its new (desired) orientation during the healing process.

The movement necessary to realign a disfigured or deformed bone often requires solving complex planning calculations as well as using a certain amount of estimation based upon the experience of the orthopedic surgeon. To aid in the accuracy of this process, several types c Computer-Aided Orthopedic Surgery (CAOS) are currently being developed. In general, CAOS involves a three step process: (1) generating a three-dimensional (3D) computerized model of the patient's bone; (2) performing a computer-aided pre-surgical analysis to generate a surgical plan that instructs a surgeon how to cut, fill, and/or reposition the bone a: well as how to manipulate a robot during surgery; and (3) performing computer-aided surgery based on the pre-surgical plan.

The current methods of modeling an incorrectly aligned bone often include the use of Magnetic Resonance Imaging (MRI) or Computerized Axial Tomography (CAT) data. These imaging technologies are very expensive and may take an extensive amount of time for which to model a bone. Conventional CAOS methods often include robot-guided surgery or real-time tracking systems using highly technical equipment reserved for a few select surgeons in a very few locations. Therefore, a need has been recognized to provide the accuracy benefits of CAOS in a more cost effective, easy to use, and more widely available process than a conventional CAOS procedure. This improved CADS process if preferably available to a wider body of patients and surgeons spread across a greater geographic and economic spectrum than current methods.

### SUMMARY

The present invention contemplates a method of generating a computer-based 3D (three dimensional) model for a patient's anatomical part comprising defining a 3D template model for the patient's anatomical part; receiving a plurality of 2D (two dimensional) images of the patient's anatomical part; extracting 2D fiducial geometry of the patient's anatomical part from each of said plurality of 2D images; and deforming the 3D template model using the 2D fiducial geometry of the patient's anatomical part so as to minimize an error between contours of the patient's anatomical part and those of the deformed 3D template model.

A computer assisted orthopedic surgery planner software according to the present invention may identify the 2D fiducial geometry of a patient's bone (or other anatomical part under consideration) on the 3D template bone model prior to deforming the 3D template bone model to substantially conform to the contours of the actual patient's bone. In one embodiment, after detecting the bone contour, the computer assisted orthopedic surgery planner software creates a 3D lattice in which the 3D template bone model is embedded. Thereafter, a free-form deformation process is applied to the 3D lattice to match with the contour of the patient's bone, deforming the 3D template bone model in the process. Sequential quadratic programming (SQP) techniques may be used to minimize error between 2D images data and the deformed template bone data.

In an alternative embodiment, a template polygonal mesh representing a standard parametric geometry and topology of a bone is defined. The template polygonal mesh is then converted into a deformable model consisting of a system of stretched springs and bent springs. Then, multiple images of the patient's bone are used to generate force constraints that deform and resize the deformable model until the projections of the deformed bone model conform to the input images. To further assist the bone geometry reconstruction problem, a standard library of image processing routines may be used to filter, threshold and perform edge detection to extract two-dimensional bone boundaries from the images.

In another embodiment, the present invention contemplates a computer-based method of generating a surgical plan comprising reading digital data associated with a 3D (threedimensional) model of a patient's bone, wherein the digital data resides in a memory in a computer; and generating a surgical plan for the patient's bone based on an analysis of the digital data associated with the 3D model. A surgical planner/simulator module in the computer assisted orthopedic surgery planner software makes a detailed surgical plan using realistic 3D computer graphics and animation. The simulated surgical plan may be viewed on a display screen of a personal computer. The planner module may also generate a pre-surgery report documenting various aspects of the bone surgery including animation of the bone distraction process, type and size of fixator frame and its struts, a plan for mounting the fixator frame on the patient's bone, the location of the osteotomy/coricotomy site and the day-by-day length adjustment schedule for each fixator strut.

In a still further embodiment, the present invention contemplates an arrangement wherein a computer assisted orthopedic surgery planner computer terminal is connected to a remote operation site via a communication network, e.g., the Internet. The computer assisted orthopedic surgery planner software may be executed on the computer assisted orthopedic surgery planner computer. A fee-based bone distraction planning (BDP) service may be offered via a network (e.g., the Internet) using the computer assisted orthopedic surgery planner software at the service provider's site. An expert surgeon at the service provider's site may receive a patient's 2D image data and other additional information from a remotely-located surgeon who will be actually operating on the patient. The remotely-located surgeon may be a subscriber to the network-based BDP service. The expert surgeon may analyze the 2D image data and other patient-specific medical data supplied by the remotely-located surgeon with the help of the computer assisted orthopedic surgery planner software executed on the computer assisted orthopedic surgery planner computer. Thereafter, the expert surgeon may send to the remotely-located surgeon over the Internet the 3D bone model of the patient's bone, a simulated surgery plan as well as a complete bone distraction schedule generated with the help of the computer assisted orthopedic surgery planner software of the present invention.

The computer assisted orthopedic surgery planner software of the present invention makes accurate surgical plans based solely on a number of two-dimensional renderings of the patient's bone geometry. The software takes into account the complex and inherently three dimensional nature of bone deformities as well as of fixator geometry. Furthermore, three dimensional simulation of the suggested surgical plan realistically portrays the complexities involved in accessing the target positions of the osteotome and fixator pins surrounding the operated bone, allowing the surgeon to accurately mount the fixator on the patient according to the pre-surgical plan.

With the computer-aided pre-operative planning and frame application and adjustment methods of the present invention, the duration of fixation (of a fixator frame) may be reduced by an average of four to six weeks. Additionally, by lowering the frequency of prolonged fixations, substantial cost savings per patient may be achieved. Shortening of the treatment time and reduction of complications may lead to better surgical results and higher patient satisfaction. The use of the computer assisted orthopedic surgery planner software of the present invention (e.g., in an Internet-based bone distraction surgery planning service) may make the frame fixation and bone distraction processes physician-friendly by simplifying fixation, decreasing preoperative planning time, and reducing the chances of complications through realistic 3D simulations and bone models. Thus more surgeons may practice bone distraction, resulting in benefits to more patients in need of bone distraction.

The present invention contemplates, in at least one preferred embodiment, devices and methods for computer-aided orthopedic surgery. More specifically, the present invention contemplates devices and methods for performing computer-aided surgical procedures, such as an open wedge osteotomy, using intra-operative feedback to improve the surgical outcome for the patient.

In at least one preferred embodiment of the present invention, a computer database includes one or more template bone models. Multiple images of an incorrectly aligned bone are preferably taken and used to “morph” or modify a stored template bone model to create a 3D model of the misaligned bone. A computer program, running on a planning computer, may be used to aid in the generation of a pre-surgical plan for performing an osteotomy or other orthopedic surgery to correct bone alignment. The pre-surgical plan calculations may include: the positioning of multifunctional markers on the patient's bone and the parameters for manipulating one or more surgical tools such as an adjustable cutting guide, an adjustable fixation guide, or a combined cutting-fixation guide.

During surgery, a surgeon preferably affixes multifunctional markers to the misaligned bone according to the pre-surgical plan. A new set of fluoroscopic or X-ray images may be taken and used by the planning computer to update the pre-surgical plan into a final surgical plan based on the actual marker positions as depicted in the fluoroscopy. In this way the updated fluoroscopic or X-ray images act as an intra-operative feedback system.

The surgeon preferably follows the updated surgical plan to cut the bone guided by an adjustable cutting guide and reposition the bon using an adjustable fixation guide (or these guides could be combined) Additionally, for example, in an open wedge osteotomy, the gap between cut sections of the bone are filled by bone graft and a fixation plate is attached thereto to hold the bone in its new orientation.

In at least one preferred embodiment, the planning compute exists at or near the same location as the surgical operating room. In other embodiments, the planning computer, template bone model database, operating room, and any other possible computers or devices may be located remotely from each other. These devices are preferably connected electronically, e.g., by way of the Internet. Such a distributed network allows access to the computer-aided osteotomy resources by an increased number of patients and surgeons than conventional methods. For example, this distributed system may be used to remotely access other experts, such as experienced orthopedic surgeons, during the planning or surgical stages.

These and other details, objects, and advantages of the present invention will be more readily apparent from the following description of the presently preferred embodiments.

### BRIEF DESCRIPTION OF DRAWINGS

Further advantages of the present invention may be better understood by referring to the following description taken in conjunction with the accompanying drawings, in which:

FIG. 1 illustrates a prior art Ilizarov fixator attached to a bone;

FIG. 2 depicts an exemplary setup to perform computer assisted orthopedic surgery according to the present invention;

FIG. 3 shows an exemplary operational block diagram for the three modules constituting the computer assisted orthopedic surgery planner software according to the present invention;

FIG. 4 graphically illustrates exemplary computer screen displays generated upon execution of the computer assisted orthopedic surgery planner software of the present invention;

FIG. 5 is an exemplary flowchart depicting operational steps performed by the 3D geometry reconstructor module of the computer assisted orthopedic surgery planner software;

FIG. 6 shows front and side X-ray images of a bone and corresponding bone boundaries extracted therefrom;

FIG. 7 portrays intersection of swept bone boundaries shown in FIG. 6;

FIG. 8 displays an undeformed 3D template bone model with the patient's bone geometry reconstructed thereon;

FIG. 9A shows free-form deformation parameters and lattices deformed according to the contour of the patient's bone;

FIG. 9B illustrates a binary tree subdivision process on a control block;

FIG. 10 illustrates a template triangular mesh in a physical-based approach to bone geometry reconstruction;

FIG. 11 illustrates extension springs and torsion springs defined over a deformable triangular mesh model;

FIG. 12 depicts the deformed 3D geometric model and the deformed lattice for the patient's bone;

FIG. 13A depicts the initial error between an X-ray image and a deformed template bone generated using a three-cell lattice;

FIG. 13B depicts the initial error between an X-ray image and a deformed template bone generated using an eight-cell lattice;

FIG. 14A depicts the final error between the X-ray image and the deformed template bone shown in FIG. 13A;

FIG. 14B depicts the final error between the X-ray image and the deformed template bone shown in FIG. 13B;

FIG. 15 is an exemplary flowchart depicting operational steps performed by the surgical planner/simulator module of the computer assisted orthopedic surgery planner software according to the present invention;

FIG. 16 is an exemplary three-dimensional surgical simulation on a computer screen depicting a fixator, a bone model and the coordinate axes used to identify the bone's deformity and the osteotomy site;

FIG. 17 shows an example of a graphical user interface screen that allows a user to manipulate the 3D simulation shown in FIGS. 4 and 16;

FIG. 18 depicts post-surgery X-ray images of a patient's bone along with the X-ray image of the fixator mounted thereon; and

FIG. 19 illustrates an exemplary fixator ring incorporating easily identifiable and detachable visual targets.

FIG. 20 shows a typical poorly aligned bone with reference axes;

FIG. 21 is a block diagram of Computer-Aided Orthopedic Surgery (CAOS) methods including a general flow chart (21A), current methods (21B), and one embodiment of the present invention (21C);

FIG. 22 details the bone modeling process including patient bone X-ray and segmentation (22A), template bone model (22B), localized MRI (22C), and the resulting fused image (22D);

FIG. 23 details the pre-surgical planning process including the calculation of the bone cutting area (23A), bone wedge opening (23B), and placement of the multifunctional markers (23C);

FIGS. 24A, B and C detail the offset analysis of a single osteotomy procedure;

FIGS. 25A, B and C detail the offset analysis of a double osteotomy procedure;

FIG. 26 details the multifunctional marker registration process including a calibration grid (26A), fluoroscopic image during surgery (26B), and resulting updated marker position bone model (26C);

FIG. 27 details a top (27A) and isometric (27B) view of an adjustable cutting guide including exemplary surgical plan (27C) and a front view of the cutting guide mounted to the multifunctional markers (27D); and

FIG. 28 details an isometric view (28A) of an adjustable fixation guide including surgical plan (28C) and a front view of the fixation guide mounted to the multifunctional markers with attached fixation plate (28B).

### DETAILED DESCRIPTION OF PREFERRED EMBODIMENTS

FIG. 2 depicts an exemplary setup to perform computer assisted orthopedic surgery according to the present invention. A computer assisted orthopedic surgery planner computer 30 is accessible to a surgeon in a remote operation site 32 via a communication network 34. In one embodiment, the communication network 34 may be an ethernet LAN (local area network) connecting all the computers within an operating facility, e.g., a hospital. In that case, the surgeon and the computer assisted orthopedic surgery terminal 30 may be physically located in the same site, e.g., the operating site 32. In alternative embodiments, the communication network 34 may include, independently or in combination, any of the present or future wired or wireless data communication networks, e.g., the Internet, the PSTN (public switched telephone network), a cellular telephone network, a WAN (wide area network), a satellite-based communication link, a MAN (metropolitan area network) etc.

The computer assisted orthopedic surgery planner computer 30 may be, e.g., a personal computer (PC) or may be a graphics workstation. Similarly, the doctor at the remote site 32 may have access to a computer terminal (not shown) to view and manipulate three-dimensional (3D) bone and fixator models transmitted by the computer assisted orthopedic surgery planner computer 30. In one embodiment, the computer assisted orthopedic surgery planner terminal 30 may function as the surgeon's computer when the operating site includes the computer assisted orthopedic surgery planner computer 30. Each computer-the computer assisted orthopedic surgery planner computer 30 and the remote computer (not shown) at the operating site-may include requisite data storage capability in the form of one or more volatile and non-volatile memory modules. The memory modules may include RAM (random access memory), ROM (read only memory) and HDD (hard disk drive) storage. Memory storage is desirable in view of sophisticated computer simulation and graphics performed by the computer assisted orthopedic surgery planner software according to the present invention.

The computer assisted orthopedic surgery planner software may be initially stored on a portable data storage medium, e.g., a floppy diskette 38, a compact disc 36, a data cartridge (not shown) or any other magnetic or optical data storage. The computer assisted orthopedic surgery planner computer 30 may include appropriate disk drives to receive the portable data storage medium and to read the program code stored thereon, thereby facilitating execution of the computer assisted orthopedic surgery planner software. The computer assisted orthopedic surgery planner software, upon execution by the computer assisted orthopedic surgery planner computer 30, may cause the computer assisted orthopedic surgery computer 30 to perform a variety of data processing and display tasks including, for example, display of a 3D bone model of the patient's bone on the computer screen 40, rotation (on the screen 40) of the 3D bone model in response to the commands received from the user (i.e., the surgeon), transmitting the generated 3D bone model to the computer at the remote site 32, etc.

Before discussing how the computer assisted orthopedic surgery planner software generates 3D bone and fixator models and simulates surgical plans for bone distraction, it is noted that the arrangement depicted in FIG. 2 may be used to provide a commercial, network-based bone distraction planning (BDP) service. The network may be any communication network 34, e.g., the Internet. In one embodiment, the surgeon performing the bone distraction at the remote site 32 may log into the BDP service provider's website and then send X-ray images, photographs and/or video of the patient's bone along with pertinent patient history to an expert surgeon located at and operating the computer assisted orthopedic surgery computer 30. The expert surgeon may then assess the case to determine if distraction is a viable option and, if so, then use the computer assisted orthopedic surgery planner software residing on the computer assisted orthopedic surgery computer terminal 30 to help plan the distraction process. The expert surgeon may transmit the distraction plan, simulation videos and distraction schedule-all generated with the help of the computer assisted orthopedic surgery planner software according to the present invention-to the service user (i.e., the surgeon at the remote site 32). Such a network-based bone distraction planning and consultancy service may be offered to individual surgeons or hospitals on a fixed-fee basis, on a per-operation basis or on any other payment plan mutually convenient to the service provider and the service recipient.

In an alternative embodiment, the network-based bone distraction planning service may be implemented without the aid of the computer assisted orthopedic surgery planner software of the present invention. Instead, the expert surgeon at the computer assisted orthopedic surgery planner terminal 30 may utilize any other software or manual assistance (e.g., from a colleague) to efficiently evaluate the bone distraction case at hand and to transmit the response back to the surgeon or user at the remote site 32.

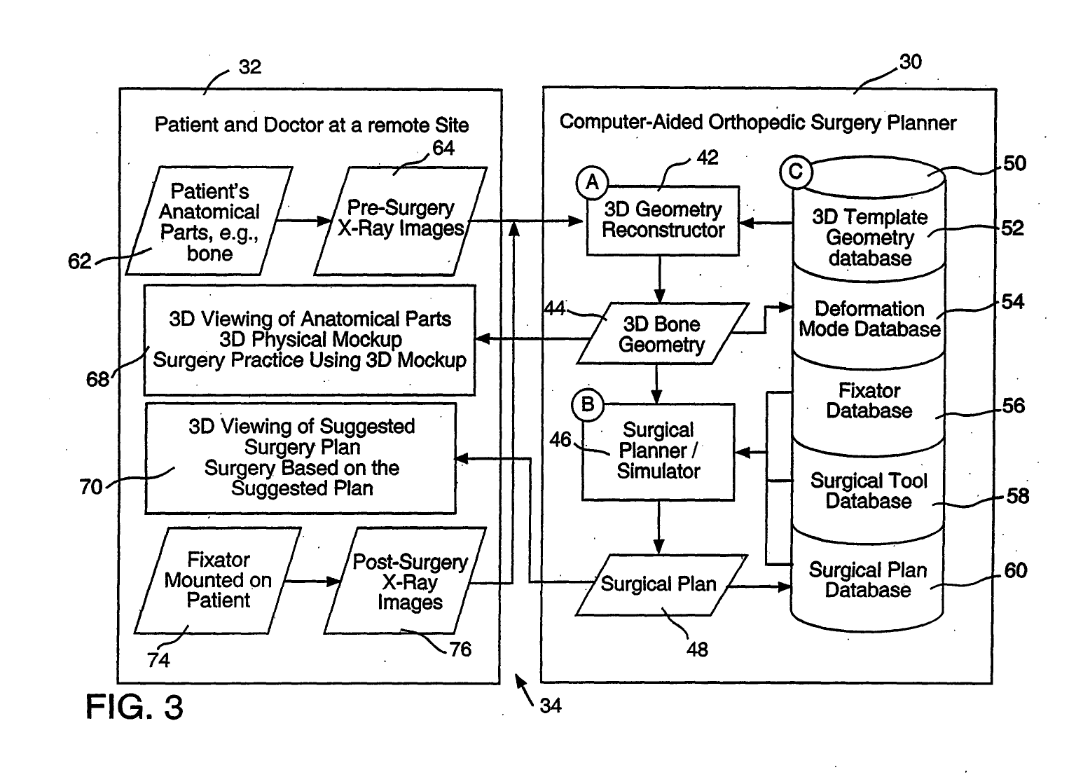

FIG. 3 shows an exemplary operational block diagram for the three modules constituting the computer assisted orthopedic surgery planner software according to the present invention. The three modules are denoted by circled letters A, B and C. Module A is a 3D geometry reconstructor module 42 that can generate a 3D bone geometry (as shown by the data block 44) from 2D (two-dimensional) X-ray images of the patient's bone as discussed hereinbelow. Module B is a surgical planner/simulator module 46 that can prepare a surgical plan for bone distraction (as shown by the data block 48). Finally, module C is a database module 50 that contains a variety of databases including, for example, a 3D template geometry database 52, a deformation mode database 54, a fixator database 56, a surgical tool database 58 and a surgical plan database 60. All of these modules are shown residing (in a suitable memory or storage area) in the computer assisted orthopedic surgery planner terminal 30. The discussion hereinbelow focuses on modules A, B and C; however, it is understood that these modules do not function independently of a platform (here, the computer assisted orthopedic surgery planner computer 30) that executes the program code or instructions for the respective module. In other words, the screen displays and printouts discussed hereinbelow may be generated only after the program code for a corresponding module is executed by the computer assisted orthopedic surgery planner computer 30.

The 3D geometry reconstructor module (or module A) 42 according to the present invention reconstructs three-dimensional bone geometry using free-form deformation (FFD) and sequential quadratic programming (SQP) techniques. Module A also generates relative positions and orientations of the patient's bone and the fixator mounted thereon. The surgical planner/simulator module (or module B) 46 provides a user-friendly simulation and planning environment using 3D, interactive computer graphics. Module B can show a realistic image of the bones, fixator and osteotomy/coricotomy, while the bone lengthening and deformity correction process is animated with 3D graphics. The database module (or module C) 50 aids in the measurement of the relative positions of the mounted fixator, osteotomy/coricotomy, and bones and feeds this information back into the computer assisted orthopedic surgery planner software to determine the final daily distraction schedule.

As an overview, it is noted that the 3D geometry reconstructor module 42 takes two (or more than two) X-ray images of patient's bone, wherein the X-ray images are taken from two orthogonal directions. Module A 42 starts with a predefined three-dimensional template bone shape, whose shape is clinically normal and is scaled to an average size. Module A 42 then scales and deforms the template shape until the deformed shape gives an image similar to an input X-ray image when projected onto a two-dimensional plane. Hierarchical free-form deformation (FFD) may be used to scale and deform the template bone, wherein the deformation in each deformation layer may be controlled by a number of variables (e.g., eight variables). Thus, the problem of finding the three-dimensional shape of the bone is reduced to an optimization problem with eight design variables. Therefore, one objective of module A 42 is to minimize the error, or the difference, between the input X-ray image and the projected image of the deformed template shape. SQP (sequential quadratic programming) techniques may be used to solve this multi-dimensional optimization problem. In other words, SQP techniques may be applied to calculate optimized FFD parameters for least error.

Generation of a 3D model of a patient's bone (or any other anatomical part) based on two or more X-ray images of the bone allows for efficient pre-, intra-, and post-operative surgical planning. It is noted that X-ray image-based shape reconstruction (e.g., generation of 3D models of an anatomical part) is more computationally efficient, cost effective and portable as compared to image processing using standard three-dimensional sensor-based methods, such as MRI (magnetic resonance imaging) or CAT (computerized axial tomography). The three-dimensional shapes generated by Module A 42 may be useful in many applications including, for example, making a three-dimensional physical mockup for surgery training or importing into and using in a computer-aided planning system for orthopedic surgery including bone distraction and open/closed wedge osteotomy. Furthermore, module A may reconstruct the 3D geometric model of the bone even if there are partially hidden bone boundaries on X-ray images.

Using CAT or MRI data for reconstructing bone geometry, however, has several practical limitations. First, compared to X-ray images, CAT and MRI are not cost or time effective, which may inhibit widespread clinical usage. X-ray imaging is available not only in large medical institutes, but also in smaller medical facilities that cannot afford CAT or MRI equipment. Second, X-ray imaging is portable so that it can be used in a remote site, even in a battlefield. In addition, the cost of scanning each patient using CAT or MRI is high, and the procedure is time consuming. Another disadvantage of using MRI or CAT is associated with the robustness of the software that performs surface geometry extraction. CAT or MRI's volumetric data has a much lower resolution compared to X-ray images, and the surface extraction process often cannot be completed due to the low resolution. Finally, X-ray imaging is preferred for imaging osseous tissues.

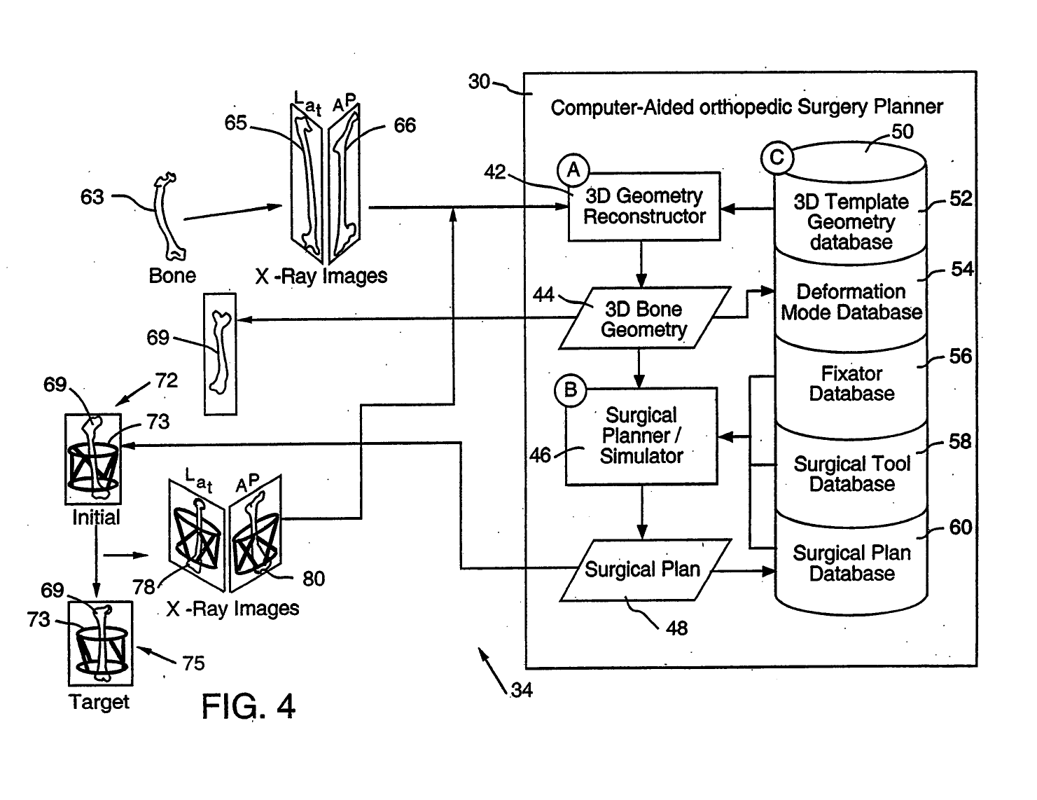

Because there is an unknown spatial relationship between the pre-operative data (e.g., medical or X-ray images, surgical plans, etc.) and the physical patient on the operating room table, the 3D geometry reconstructor module 42 provides for both pre-operative and intra-operative registration of orthopedic bone deformity correction. A 3D solid model of the bone generated by module A 42 (as shown by data block 44 in FIG. 3 and 3D bone image 67 in FIG. 4) may function as a fundamental tool for pre-, intra-, and post-operative surgical planning. The 3D geometry reconstructor module 42 develops interactive, patient-specific pre-operative 3D bone geometry to optimize performance of surgery and the subsequent biologic response.

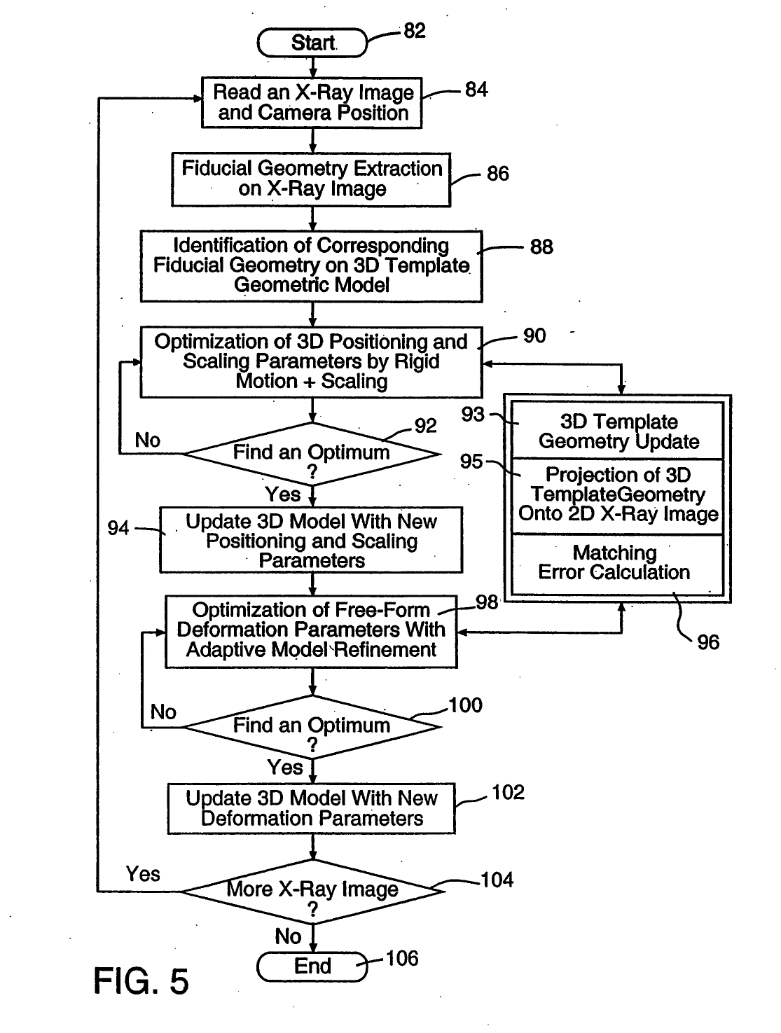

FIG. 4 graphically illustrates exemplary computer screen displays generated upon execution of the computer assisted orthopedic surgery planner software of the present invention. FIGS. 3 and 4 may be viewed together to better understand the functions performed by modules A, B and C, and also to have a visual reference of various 3D models generated by the computer assisted orthopedic surgery planner software according to the present invention. Furthermore, FIG. 5 is an exemplary flowchart depicting operational steps performed by the 3D geometry reconstructor module 42 of the computer assisted orthopedic surgery planner software. The following discussion will also refer to various operational steps in FIG. 5 as appropriate.

Initially, at block 62, a surgeon determines (at a remote site 32) which of the patient's anatomical parts (e.g., a bone) is to be operated on. FIG. 4 shows a bone 63 that is to be distracted. Thereafter, at block 64, the surgeon or an assistant of the surgeon prepares digitized X-ray images for various X-ray views of the patient's bone 63. Digitization may be carried out manually, e.g., by placing an X-ray image on a light table and then tracing the outline of the bone contour with a digitizing stylus. In the embodiment illustrated in FIG. 4, digitized versions of a lateral (Lat) X-ray image 65 and an anterior/posterior (AP) X-ray image 66 of the bone 63 are input to the computer assisted orthopedic surgery planner software via the communication network 34 interconnecting the remote patient site 32 and the computer assisted orthopedic surgery planner terminal 30. It is noted that the X-ray images 65,66 represent bone geometry in two-dimensional (2D) views.

Upon execution of module A (at step 82 in FIG. 5), module A 42 receives (at block 84 in FIG. 5) as input the digitized X-ray images 65,66. It is assumed that the X-ray images 65,66 are taken from two orthogonal directions, usually front (or AP) and side (or lateral). This constraint of the orthogonal camera positions is a strong one, but it may be loosened, if necessary, with the modification of deformation parameters and extra computational cost in the optimization process. Module A 42 may also receive positional data for the X-ray camera (not shown) with reference to a pre-determined coordinate system. Such coordinate position may be useful for module A 42 to “read” the received X-ray images 65,66 in proper geometrical context. A user, e.g., the operator of the X-ray camera, may manually input the camera position coordinates and viewing angle data. Alternatively, a scheme may be devised to automatically incorporate the camera position parameters and viewing angle data as a set of variables to be optimized during the optimization process discussed hereinbelow. More than two X-ray images could be added to the input if greater accuracy is required or if a certain part of the bone that is hidden in the AP and lateral views plays an important role in the bone distraction procedure. Since MRI and CAT have volumetric data set, using X-ray images to reconstruct the bone structure (e.g., the 3D geometric module 69) is more cost-effective and less time-consuming.

After receiving the 2D X-ray images 65,66, the 3D geometry reconstructor module 42 may extract at step 86 the fiducial geometry (or bone contour) from the X-ray images. The 2D X-ray images 65,66 represent the bone contour with a set of characteristic vertices and edges with respect to the respective X-ray image's coordinate system. In one embodiment, an operator at the computer assisted orthopedic surgery planner terminal 30 may manually choose (with the help of a keyboard and a pointing device, e.g., a computer mouse) the bone contour from the 2D X-ray images 65,66 of the bone 63 displayed on the computer screen 40. In another embodiment, commercially available edge detection software may be used to semi-automate the fiducial geometry extraction process.

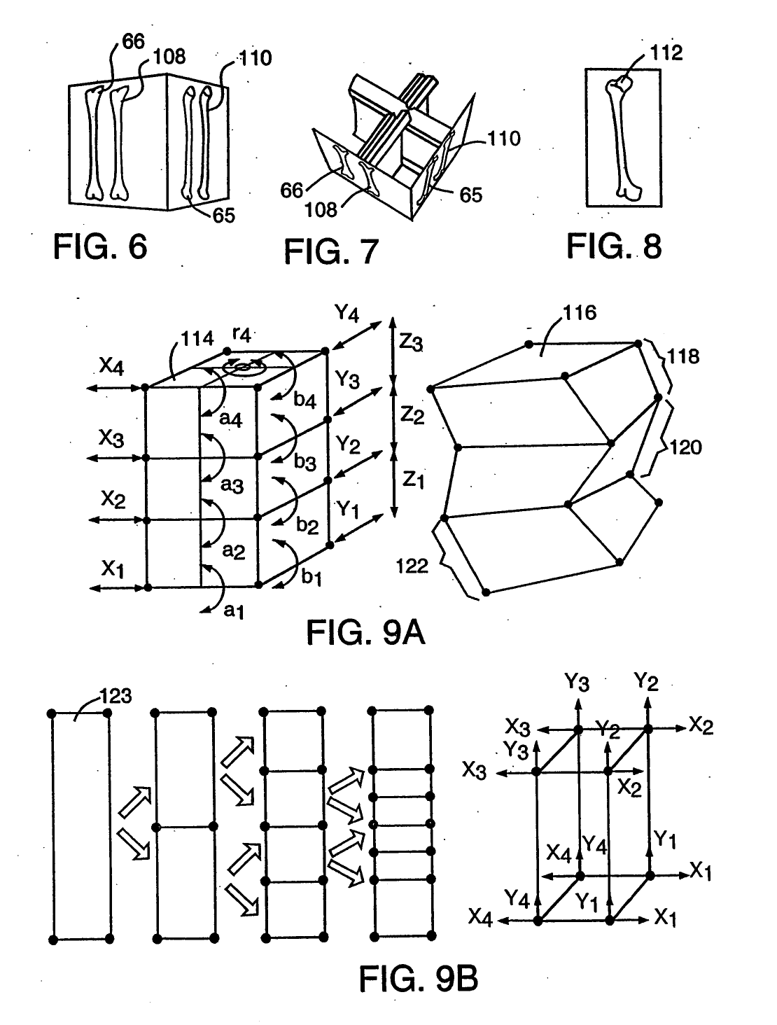

After, before or simultaneous with the fiducial geometry extraction, module A 42 may access the 3D template geometry database 52 to select a 3D template bone model (not shown) that may later be deformed with the help of the 2D X-ray images 65,66 of the patient's bone 63. The size (or outer limits) of the 3D template bone model may be selected based on the computation of the closed volume that tightly bounds the patient's bone geometry. FIGS. 6 and 7 illustrate certain of the steps involved in that computation. FIG. 6 shows front (66) and side (65) X-ray images of a bone and corresponding bone boundaries (108 and 110 respectively) extracted therefrom. FIG. 7 portrays the intersection of swept bone boundaries 108, 110 shown in FIG. 6. The intersection of the bone boundaries defines a closed volume that may tightly bound the 3 D template bone model and that closely resembles the volumetric dimensions of the patient's bone.

After detecting the bone contour at step 86, module A 42 first identifies (at step 88) the corresponding fiducial geometry on the 3 D template bone model prior to any deformation discussed hereinbelow. Module A 42 also optimizes (at steps 90 and 92) the 3D positioning and scaling parameters for the 3D template bone model until the size and position of the 3D template bone model is optimum with respect to the patient's bone 63 (as judged from the X-ray images 65,66 of the patient's bone 63). Upon finding the optimum values for positioning and scaling parameters, module A 42 updates (at step 94) the 31) template bone model with new positioning and scaling parameters. The resultant 3D template bone model 112 is shown in FIG. 8, which displays the undeformed 3D template bone model 112 with the patient's bone geometry reconstructed thereon. Module A 42 may also update (block 93) the 3D template geometry database 52 with the optimum positioning and scaling parameter values computed at steps 90 and 92 for the selected template bone model. Thus, the 3D template geometry database 52 may contain 31) template bone models that closely resemble actual, real-life patients' bones.

In one embodiment, the 3D geometry reconstructor module 42 creates a 3D lattice 114 in which the template bone 112 from FIG. 8 is embedded. A free-form deformation process is applied to this 3D lattice 114 in order to optimally match with the contour of the patient's bone. For the sake of simplicity, a few of the free-form deformation (FFD) parameters are shown in FIG. 9A and identified as ai, bi, and ri (where i=1 to 4) in the x-y-z coordinate system for each parallelpiped (118, 120 and 122) in the 3D lattice 114. It may be desirable to have the 3D lattice 114 watertight in the sense that there may not be any gap and overlap between the faces of each constituent parallelpiped (118, 120 and 122) so as not to adversely affect a physical mockup made with a rapid prototyping process. In one embodiment, Sederberg and Parry's technique (hereafter “Parry's technique”) may be used to reconstruct three-dimensional geometric model of the patient's bone. A detailed description of Parry's technique may be found in T. W. Sederberg and S. R. Parry, “Free Form Deformation of Solid Geometric Models,” presented at SIGGRAPH '86 Proceedings, Dallas, Tex. (1986), which is incorporated herein by reference in its entirety.

It is stated in A. H. Barr (hereafter “Barr”), “Global and Local Deformations of Solid Primitives,” Computer Graphics, vol. 18, pp. 21-30 (1984), which is incorporated herein by reference in its entirety, that “Deformations allow the user to treat a solid as if it were constructed from a special type of topological putty or clay which may be bent, twisted, tapered, compressed, expanded, and otherwise transformed repeatedly into a final shape.” Barr uses a set of hierarchical transformations for deforming an object. This technique includes stretching, bending, twisting, and taper operators. However, Parry's technique deforms the space (e.g., the parallelpiped 3D lattice 114 in FIG. 9A) in which the object is embedded (as shown in FIG. 12). On the other hand, Coquillart's Extended Free-Form Deformation (EFFD) technique changes the shape of an existing surface either by bending the surface along an arbitrarily shaped curve or by adding randomly shaped bumps to the surface using non-parallelpiped type 3D lattices as discussed in S. Coquillart, “Extended Free-Form Deformation: A Sculpturing Tool for 3D Geometric Modeling,” Computer Graphics, vol. 24, pp. 187-196 (1990) and in S. Coquillart, “Extended Free-Form Deformation: A Sculpturing Tool for 3D Geometric Modeling,” INRIA, Recherche, France 1250 (June 1990), both of these documents are incorporated herein by reference in their entireties.

Here, Parry's FFD technique is applied to a new area of application, i.e., three-dimensional shape reconstruction from two-dimensional images, instead of to the traditional application domains of geometric modeling and animation. Additionally, hierarchical and recursive refinement is applied to the control grid of FFD to adjust the deformation resolution. Hierarchical refinement may be necessary because of the unique nature of the shape reconstruction problem, i.e., lack of a priori knowledge of the complexity or severity of the deformation.

The basic idea of Parry's technique is that instead of deforming the object (here, the 3D template bone) directly, the object is embedded in a rectangular space that is deformed (as illustrated by FIG. 12). One physical and intuitive analogy of FFD is that a flexible object may be visualized as being “molded” in a clear plastic block and the whole block is deformed by stretching, twisting, squeezing, etc. As the plastic block is deformed, the object trapped inside the block is also deformed accordingly. Parry's technique uses the following single Bezier hyperpatch to perform this deformation:

$$q ( u , v , w ) = {\sum }_{i = 0}^{n} {\sum }_{j = 0}^{n} {\sum }_{k = 0}^{n} {P}_{ijk} {B}_{i} ( u ) {B}_{j} ( v ) {B}_{k} ( w ) , 0 \leq u \leq 1 , 0 \leq v \leq 1 , 0 \leq w \leq 1 \qquad ( 1 )$$

where u, v, and w are parameter values that specify the location of an original point in the control block space, q(u, v, w) specifies the location of the point after the deformation, Pᵢⱼₖ specifies points that define a control lattice, and Bᵢ(u), Bⱼ(v), and Bₖ(W) are the Bernstein polynomials of degree n, for example:

$${B}_{i} ( u ) = \frac{n !}{i ! ( n - i ) !} {{u}^{i} ( 1 - u )}^{n - i} \qquad ( 2 )$$

In equation (2), a linear version of FFD as a unit deformation block (i.e., n=1) may be used. This is the simplest deformation function, and there are only eight control points used to define a control block for deformation-these eight points define eight corner points of a deformation block (e.g., as shown by the corner points of each parallelpiped in the 3D lattice 114 in FIG. 9A). The variation of a deformation with a linear function is limited compared to a higher order function, but a linear function may be preferable because the complexity of the deformation of a bone is unknown a priori. It may also be desirable to increase the resolution of a deformation as needed by using adaptive refinement of the control block.

The adaptive refinement may be performed by using a hierarchical, recursive binary tree subdivision of the control block 123 as shown in FIG. 9B. A binary tree subdivision may be preferable rather than a more standard spatial subdivision of octree subdivision, because of the cylindrical or rim-type shape of the target bones (i.e., bones to be operated on) of a human patient. Octree may be a better choice when the target bone shape is not cylindrical. Furthermore, the extension from a binary subdivision to an octree subdivision may be straightforward.

Parry's technique calculates the deformed position X_{ffd} of an arbitrary point X, which has (s, t, u) coordinates in the system given by the following equation:

X=Xₒ+sS+tT+uU

The (s, t, u) coordinates are computed from the following equations:

$$\begin{matrix}s = \frac{T \times U \cdot ( X - {X}_{0} )}{T \times U \cdot S} & ( 4 ) \\ t = \frac{S \times U \cdot ( X - {X}_{0} )}{S \times U \cdot T} & ( 5 ) \\ u = \frac{S \times T \cdot ( X - {X}_{0} )}{S \times T \cdot U} & ( 6 )\end{matrix}$$

A grid of the control points, Pᵢⱼₖ in equation (7) is imposed on each parallelpiped (118, 120 and 122). This forms l+1 planes in the S direction, m+1 planes in the T direction, and n+1 planes in the U direction.

$${P}_{ijk} = {X}_{0} + \frac{i}{l} S + \frac{j}{m} T + \frac{k}{n} U \qquad ( 7 )$$

The deformation is then specified by moving the Pᵢⱼₖ from their undisplaced, lattical positions according to the following equation:

$${X}_{ffd} = {\sum }_{i = 0}^{l} ( \begin{matrix}l \\ i\end{matrix} ) {( 1 - s )}^{l - i} {s}^{i} [ {\sum }_{j = 0}^{m} ( \begin{matrix}m \\ j\end{matrix} ) {( 1 - t )}^{m - j} {t}^{j} [ {\sum }_{k = 0}^{n} ( \begin{matrix}n \\ k\end{matrix} ) {( 1 - u )}^{n - k} {u}^{k} {P}_{ijk} ] ] \qquad ( 8 )$$

A sequential quadratic programming (SQP) algorithm may then be used to compute free form deformation (FFD) parameters (aᵢ, bᵢ and rᵢ in FIG. 9A) that minimize the error between the X-ray image and the deformed bone image. Because the 3D geometry reconstructor module 42 creates three connected parallelpipeds (118, 120 and 122 in FIG. 9A), there are a total of eight parameters subject to optimization. More accuracy (i.e., minimization of error) may be achieved with increasing the number of parallelpiped lattices and also by increasing the number of FFD parameters. Before calculating the error, module A 42 may shrink the template bone data and the X-ray image data into a unit cube for convenient computation. The objective function of this minimization problem can be defined as follows:

$$\sum \vert {P}_{n} - {Q}_{n} ( {a}_{1} , {a}_{2} , … ) \vert \qquad ( 9 )$$

where Pₙ represents points on the boundary of an X-ray image; Qₙ represents points on the deformed bone template; and a₁, a₂, etc. represent all deformation parameters (i.e., aᵢ, bᵢ and rᵢ in FIG. 9A). If there is no error between the X-ray image under consideration and the deformed bone image, and if the X-ray image is perfectly oriented, then the objective function in equation (9) above becomes zero.

Steps 95-102 in FIG. 5 depict the process of optimizing the FFD parameters and, hence, minimizing the error (in equation (9)h) between a corresponding 2D X-ray image (e.g., the lateral view 65 or the AP view 65 or any other available view) and the appropriate view of the 3D template bone geometry 112 projected onto that X-ray image. Module A 42 projects (at step 95) the appropriate view of the 3D template bone geometry 112 onto the corresponding 2D X-ray image (e.g., views 65 or 65 in FIG. 4) and calculates the matching error (at step 96) between the projection and the X-ray image. Based on the error calculation, module A 42 attempts to optimize the FFD parameters at steps 98 and 100. The optimized values for the FFD parameters may then be used to generate the deformed polygonal mesh 116. At step 102, the 3D template bone model 112 is updated (i.e., deformed) with the new deformed polygonal mesh 116 taking into account the new deformation parameters.

The process outlined by steps 84-102 is continued for each new X-ray image (e.g., for the lateral view 65 as well as for the AP view 65 in FIG. 4) as indicated by the decision block 104. The process terminates at step 106 and the 3D geometry reconstructor module 42 outputs the final 3D bone geometry data (block 44 in FIGS. 3 and 4) in the form of a 3D deformed bone model 69 for the patient's bone 63. The optimized values of FFD parameters obtained for a specific 3D template bone corresponding to a given bone contour (e.g., the patient's bone 63) may be stored in the deformation mode database 54 for future reference as well as to facilitate 3D viewing. The 3D solid bone model 69 may then be viewed by the surgeon at the remote site 32 for further surgical planning as depicted by block 68 in FIG. 3.

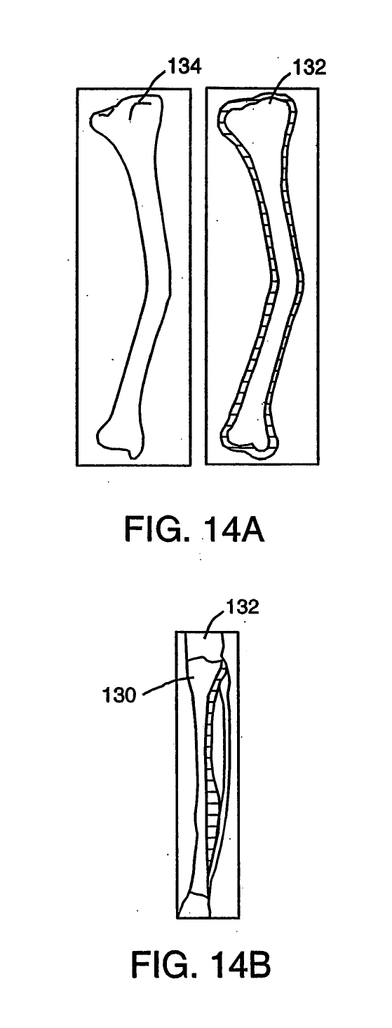

Certain of the steps discussed hereinbefore with reference to FIG. 5 are depicted in FIGS. 12, 13 and 14. FIG. 12 depicts the deformed 3D geometric model 69 and the deformed lattice 116 for the patient's bone 63. FIG. 13A depicts the initial error between an X-ray image 132 and a deformed template bone 130 generated using a lattice with three cells or three parallelpipeds (e.g., the lattice 114 in FIG. 9A). FIG. 13B, on the other hand, depicts the initial error between an X-ray image 132 and a deformed template bone 130 generated using a lattice with eight cells or eight parallelpipeds (e.g., the lattice resulting from the binary tree subdivision of the control block 123 in FIG. 9B). Due to significant errors in FIGS. 13A and 13B, the optimization process at steps 98, 100 (FIG. 5) may continue to minimize the projection error (i.e., to continue deforming the template bone 130). FIG. 14A depicts the final error between the X-ray image 132 and the deformed template bone 130 shown in FIG. 13A. In other words, FIG. 14A shows the final error in a deformation process that uses a lattice with three cells (e.g., the lattice 114 in FIG. 9A). On the other hand, FIG. 14B depicts the final error between the X-ray image 132 and the deformed template bone 130 shown in FIG. 13B. In other words, FIG. 14B shows the final error in a deformation process that uses a lattice with eight cells or eight parallelpipeds (e.g., the lattice resulting from the binary tree subdivision of the control block 123 in FIG. 9B).The eventually deformed template bone 134 may have bone geometry that closely resembles that of the patient's bone 63. The entire 3D bone model generation process depicted in FIG. 5 may be implemented in any suitable programming language, such as, e.g., the C⁺⁺ programming language, and may be executed on any suitable computer system, such as, e.g., a personal computer (PC), including the computer assisted orthopedic surgery planner computer 30. The final deformed bone geometry 69 may be displayed on the display screen 40 (FIG. 2) and may also be sent to the surgeon at the remote site 32 over the communication network 34 as discussed hereinbefore.

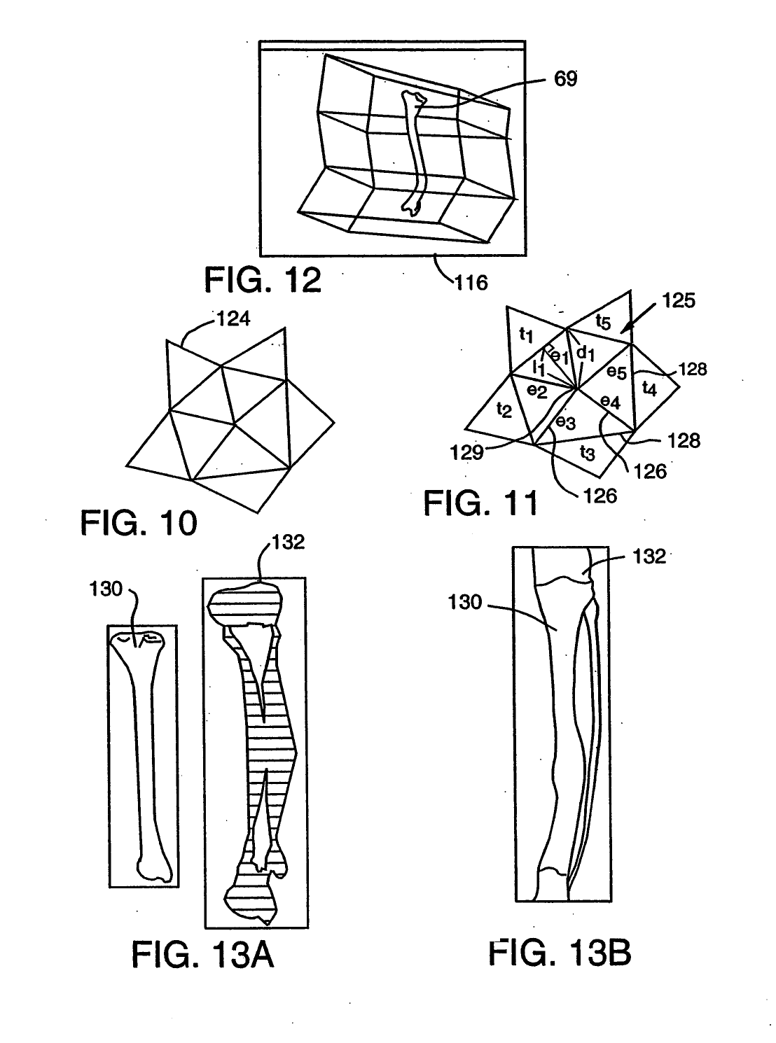

In an alternative embodiment, a physical-based approach may be used to create a 3D solid. (or deformed) template bone model (i.e., the model 69 in FIG. 4) that may later be used by the surgeon at the remote site 32 for, e.g., mockup surgery practice. As part of the deformation process, first, a template polygonal mesh that represents a standard parametric geometry and topology of a bone is defined. The length and girth of the polygonal mesh is scaled for each patient based on the size of the corresponding 3D template bone model (e.g., the 3D template bone model 112 in FIG. 8). A model consisting of parametric surfaces, such as Bezier surfaces and non-uniform rational B-spline (NURBS) surfaces may provide increased resolution. FIG. 10 illustrates a template triangular mesh 124 in a physical-based approach to bone geometry reconstruction. The contours of the 3D template bone model 112 (FIG. 8) may be visualized as being composed of the triangular mesh 124.

Thereafter, the template polygonal mesh (here, the triangular mesh 124) is converted into a deformable model consisting of a system of stretched springs and bent springs. FIG. 11 illustrates extension springs (ei) and torsion springs (ti) defined over a deformable triangular mesh model 125. Then, multiple X-ray images (e.g., images 65 and 65 in FIG. 4) are used to generate force constraints that deform and resize the deformable model 125 until the projections of the deformed bone model conform to the input X-ray images as shown and discussed hereinbefore with reference to FIGS. 13 and 14. A standard library of image processing software routines that filter, threshold and perform edge detection may be used to extract (for comparison with the projections of the deformed bone model) the two dimensional bone boundaries from the X-ray images as discussed hereinbefore.

Referring now to FIG. 11, the extension springs (ei) are defined over the edges 126 and the torsion springs (tᵢ) are defined over the edges 128 for a node 129 under consideration. It is assumed that the original length of an extension spring is given by an edge (e.g., the edge 126) of the template polygon mesh (here, the triangular mesh 125) so that the tensile force is proportional to the elongation of that edge. The spring constant of an extension spring may be denoted as ‘k’. It is also assumed that the original angle of a torsion spring is given by the template mesh (here, the mesh 125) so that the torque exerted by the torsion spring is computed based on the angular displacement. The spring constant of a torsion spring may be denoted as ‘Bᵢ’.

The total force ‘f’ exrted on a node (e.g., the center node 129) is calculated by summing: (1) the tensile forces ‘fₑᵢ’ applied by all the extension springs attached to the node, and (2) the forces ‘fⱼᵢ’ applied by all the torsion springs surrounding the node 129. In the deformable triangular mesh model 125, five extension springs eᵢ (i=1 to 5) and five torsion springs tᵢ (i=1 to 5) exert forces on the center node 129. The total force ‘f’ is thus calculated as the summation of the forces from all the springs as given by the following equation:

$$\begin{matrix}f = {\sum }_{i = 1}^{N} {f}_{{e}_{i}} + {\sum }_{i = 1}^{N} {f}_{{f}_{i}} \\ = {\sum }_{i = 1}^{N} {kd}_{i} + {\sum }_{i = 1}^{N} \frac{{β}_{i} {θ}_{i}}{{l}_{i}}\end{matrix} \qquad ( 10 )$$

where N is the number of edges attached to the node (here, the center node 129). Thus, N is equal to the number of triangles surrounding the node. Furthermore, in equation (10), dᵢ is the length of the extension spring eᵢ, θᵢ is the angle between the normal vectors of the two triangles that share the torsion spring ti as a common edge, and lᵢ is the perpendicular distance from the node (here, the center node 129) to the torsion spring tᵢ.

By defining the equation of motion of this spring system and by numerically integrating the equation of motion, an equilibrium configuration of the spring system that minimizes the potential energy of the system can be given by the following equation:

$$U = {\sum }_{all \\ nodes} ( {\sum }_{i = 1}^{N} \frac{1}{2} {kd}_{i}^{2} + {\sum }_{i = 1}^{N} \frac{1}{2} {β}_{i} {θ}_{i}^{2} ) \qquad ( 11 )$$

Thus, each triangle in the deformable triangular mesh 125 may get deformed according to the force constraints generated by the resulting mismatch (at steps 95,96 in FIG. 5) when the image of the 3D template bone geometry 112 (FIG. 8) is projected onto a corresponding 2D X-ray image (e.g., the lateral view 65, the AP view 66, etc.). The deformation of the triangular mesh 125 may continue until-the matching error is minimized as indicated by steps 96, 98,100 and 102. Upon minimization of the matching error, an equilibrium condition may get established as given by equation (11). The equilibrium process outlined above for the triangular mesh spring model of FIGS. 10 and 11 may be repeated for each X-ray image of the patient's bone 63 as denoted by the decision step 104 in FIG. 5.

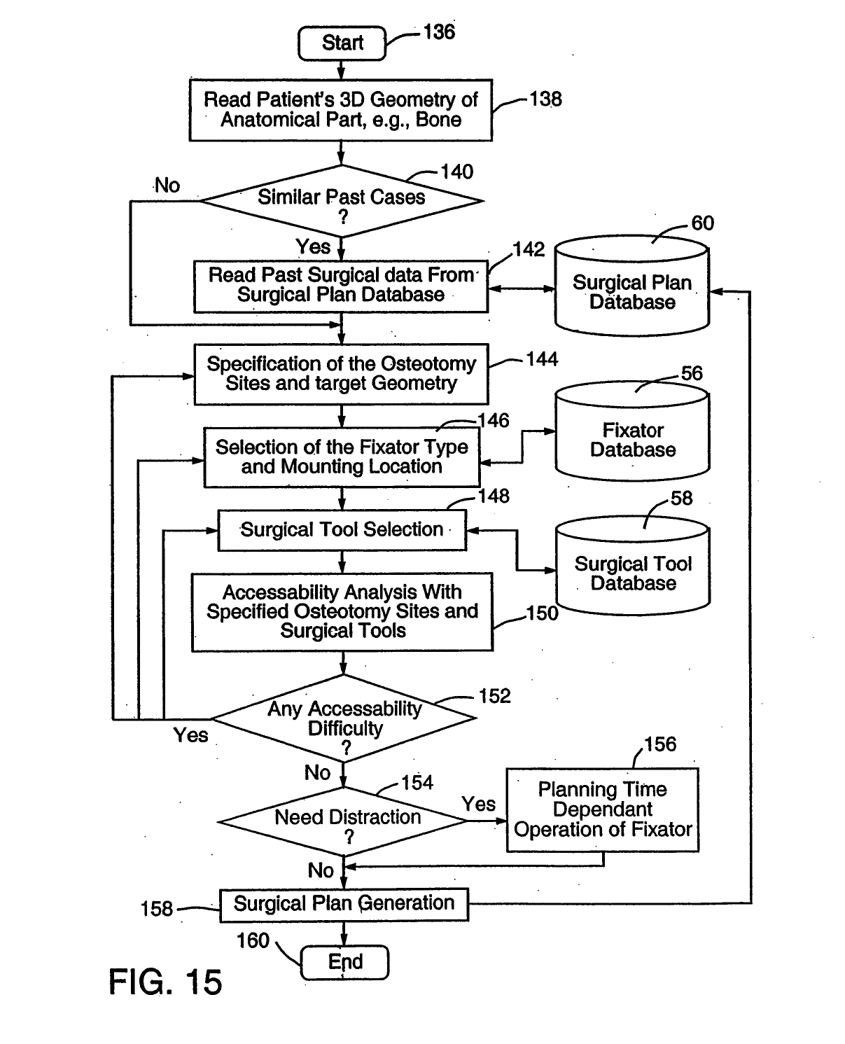

FIG. 15 is an exemplary flowchart depicting operational steps performed by the surgical planner/simulator module (or module B) 46 of the computer assisted orthopedic surgery planner software according to the present invention. Module B 46 assists a surgeon in making a detailed surgical plan by utilizing accurate 3D bone models (generated by module A 42) and realistic 3D computer graphics and animation. Upon initial execution (at step 136), the planner module 46reads or takes as an input (at step 138) the 3D geometry of the patient's anatomical part (here, the patient's bone 63). This 3D geometry may have been generated earlier by the 3D geometry reconstructor module 42 as discussed hereinbefore with reference to FIGS. 5-14. Thereafter, the surgeon viewing the 3D bone model 69 may determine (at step 140) whether any similar past case exists where the bone treated had similar 3D geometry as the current patient's bone 63. The surgeon may make the decision either upon manual review of the patient's 3D bone geometry 69 or using the surgical plan database 58 or any similar data storage. Alternatively, module B 46 may perform similar decision-making based on a comparison with the data stored in the surgical plan database 60.

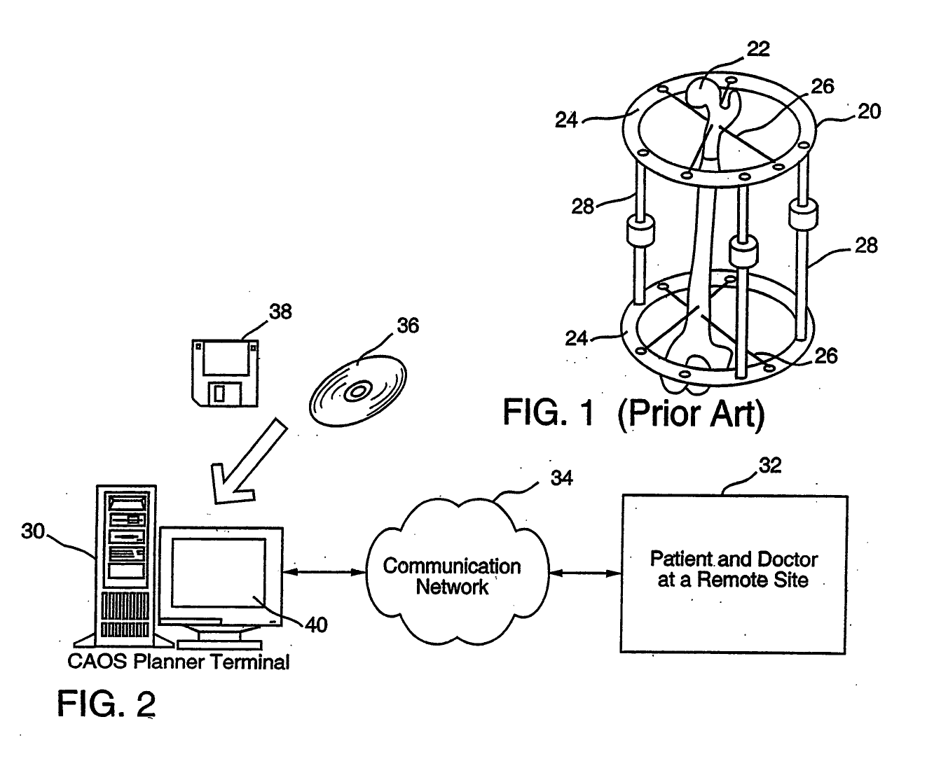

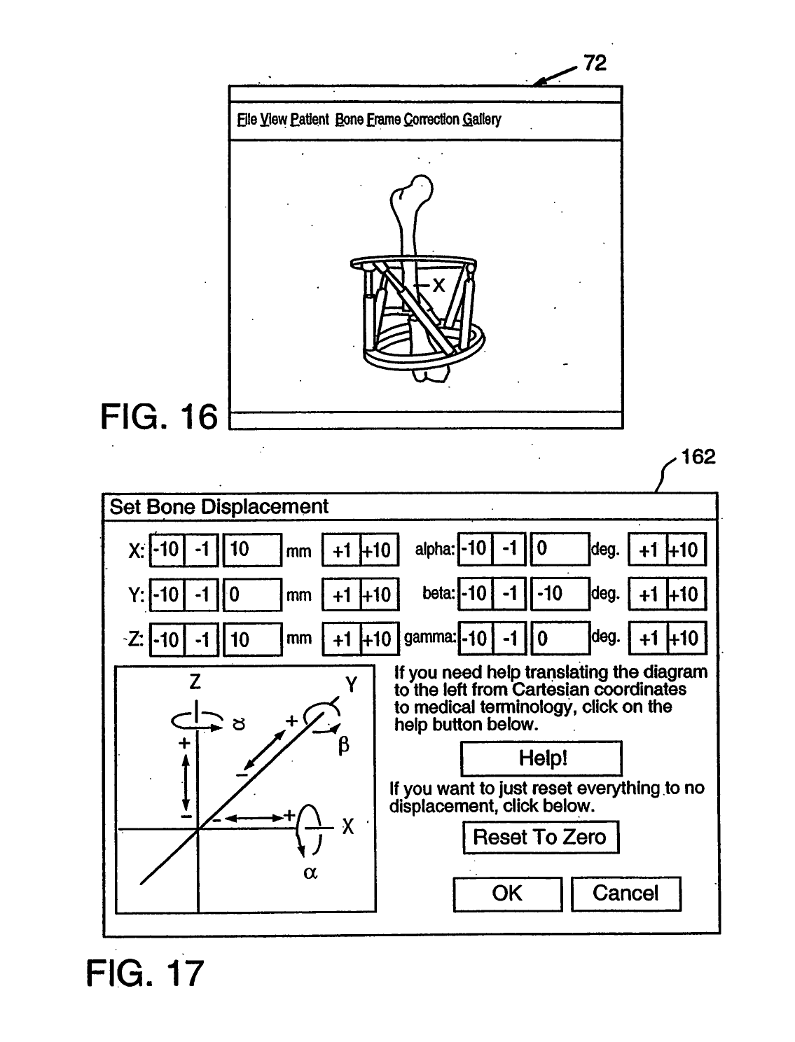

If there is a past case that involves a bone having similar 3D geometry as the current patient's bone 63, then the surgeon may instruct (at step 142) module B 46 to read the surgical data associated with the past case from the surgical plan database 60. Alternatively, upon finding a matching or similar past case, module B 46 may automatically perform a search of the surgical plan database 60 to retrieve and send pertinent past surgical data to the surgeon at the remote site 32 so that the surgeon may determine whether to follow the steps performed earlier in another case or to alter or improve the earlier executed surgical plan. Whether there is a past similar case or not, the surgical planner module 46 generates a specification of the osteotomy site(s) and of the target geometry (e.g., the mounting arrangement 75 in FIG. 4) at step 144. Thereafter, at step 146 , the planner module 46 may access the fixator database 56 to select the appropriate fixator type (e.g., the Ilizarov fixator 20 of FIG. 1 or the Taylor Spatial Frame 162 of FIG. 16). Further, during step 146, the planner module 46 may also generate information about the least intrusive mounting location for the fixator selected.

Module B (i.e., the planner module 46) may further continue the optimum and most efficient surgical plan generation process by selecting (at step 148), from the surgical tool database 58, appropriate surgical tools that may be needed to perform osteotomy or bone distraction on the patient's bone 63. Module B 46 may take into account the 3D geometry of the template bone model 69 generated by module A 42 to determine the most useful set of tools for the desired surgical procedure. The surgical planner module 46 then performs an analysis (at step 150) of how easily accessible the osteotomy site (specified earlier at step 144) is with the current selection of surgical tools (at step 148). The surgical planner module 46 may analyze (at the decisional step 152) its accessibility determination at step 150 based on, for example, an earlier input by the surgeon as to the kind of surgery to be performed on the patient's bone 63 and also based on the contour data available from the 3D template bone geometry generated by module A 42. If the planner module 46 determines any difficulty (e.g., difficulty in mounting the fixator or difficulty in accessing the osteotomy site, etc.) with the currently determined accessibility approach, then the planner module 46 may reevaluate its earlier determinations as shown by the iteration performed at step 152.

Upon determining a viable (i.e., easily accessible and least intrusive) surgical plan for the patient's bone 63, the planner module 46 may further prepare a time-line for the bone distraction operation (at step 156) based on a decision at step 154. The surgeon at the remote site 32 may specify prior to executing the computer assisted orthopedic surgery planner software whether bone distraction needs to be performed and whether the surgeon would like to have a computer-based time-line for the distraction process (including such steps as fixator mounting, daily adjustment of struts and final removal of the fixator). Finally, at step 158, the planner module 46 generates an optimum surgical plan 48 (FIGS. 3 and 4) for the patient's bone 63 based on available bone geometry and other surgical data. Prior to ending at step 160, module B 46 may store the recommended surgical plan in the surgical plan database 60 for future reference (e.g., for case comparison in a future case) and may also send the plan 48 to the surgeon at the remote site 32 via the communication network 34. In one embodiment, the surgical plan 48 may include a report documenting: (1) animation of the bone distraction process, (2) type and size of the fixator frame and its struts, (3) a suggested fixator frame mounting plan, (4) the osteotomy/coricotomy site location, (5) locations of fixator pins, and (6) the day-by-day length adjustment schedule for each fixator strut.

The surgeon at the remote site 32 may view the suggested surgery plan 48 received from the computer assisted orthopedic surgery planner computer 30 as depicted by block 70 in FIG. 3. The realistic 3D computer graphics and animation contained in the simulated surgery plan create a CAD (computer aided design) environment that can help a surgeon better understand the three-dimensional positional relationships between the bone, the fixator, the osteotomy/coricotomy site, and the fixator pins. Because the surgeon would be able to create and verify the operation plan using easy-to-understand three-dimensional views, a more precise plan could be made in a shorter period of time. In one embodiment, the three-dimensional graphics for the surgical plan 48 may be generated using the OpenGL (open graphics library) software interface developed by Silicon Graphics, Inc., of Mountainview, Calif., USA. The OpenGL graphics software interface may be implemented on a conventional PC (personal computer) platform to show animations of the bone distraction process.

The 3D simulation of the proposed surgical plan is depicted as the initial simulation 72 in FIG. 4. The computer-assisted surgical simulation 72 depicts the 3D template bone geometry 69 for the patient's bone 63 with a Taylor Spatial Frame 73 mounted thereon according to the specifications computed by module B 46 . The final location and orientation of the fixator frame 73 on the 3D solid bone model 69 is depicted by the simulated target position 75 in FIG. 4. Thus, the initial operational position 72 and the final or desired target position 75 are simulated by the surgical planner module 46 to guide the surgeon during the actual surgery.

FIG. 16 also shows the initial three-dimensional surgical simulation 72 on a computer screen depicting the fixator 73, the 3D solid bone model 69 and the coordinate axes used to identify the bone's deformity and the osteotomy site. The location of the suggested cutting of the bone for the bone distraction is also visible in the 3D simulated model 72 in FIG. 16.

FIG. 17 shows an example of a graphical user interface (GUI) screen 162 that allows a user (e.g., a surgeon) to manipulate the 3D simulations 72 or 75 shown in FIGS. 4 and 16. Thus, the surgeon at the remote site 32 may manipulate the 3D simulated models 72 or 75 with a pointing device (e.g., a computer mouse) and through the Microsoft Windows® dialog box (or GUI) 162 appearing on the screen of the computer where the surgeon is viewing the 3D models. Using the dialog box or the GUI 162 the surgeon may correct the stress-tension for the struts in the fixator frame 73 and view the simulated results prior to actually attempting the surgery.

The surgeon may then perform the surgery as suggested by the surgical plan generated by the computer assisted orthopedic surgery planner software module B 46. X-ray imaging is again used to measure all the relative positions after the fixator frame (e.g., the Taylor Spatial Frame 73) has been actually mounted (at block 74 in FIG. 3) and after the osteotomy/coricotomy has been made by the surgeon. A computer-aided surgery module may measure the actual positions of the bone deformity relative to the attached fixator and coricotomy, and the surgeon at the remote site 32 may feedback or input the positional data generated by such measurement into the computer assisted orthopedic surgery planner software for final determination of the distraction schedule based on the actual surgical data. The feedback data from the actual surgery may be sent to the computer assisted orthopedic surgery planner computer 30 over the communication network 34 as shown by the post-surgery X-ray images data output from block 76 in FIG. 3.

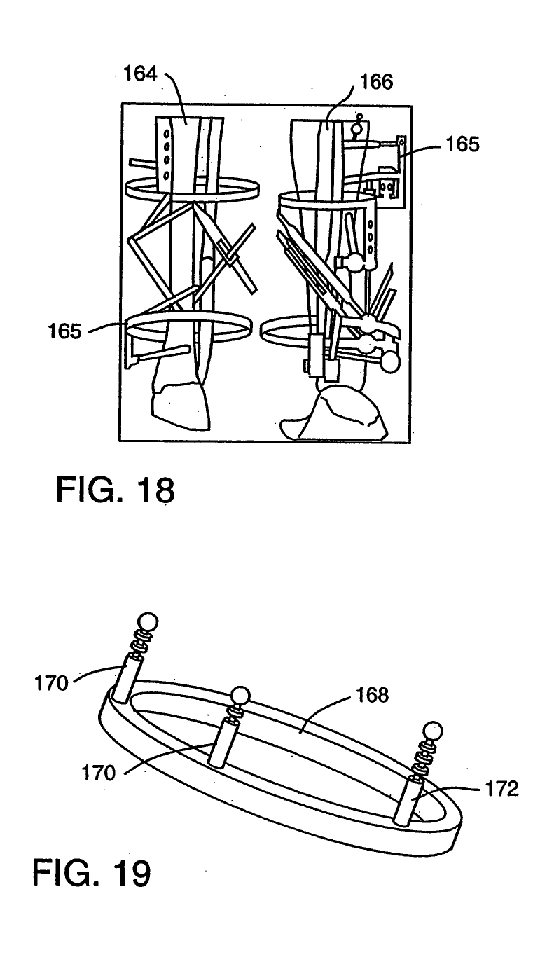

FIG. 18 depicts post-surgery X-ray images (164, 166) of a patient's bone along with the X-ray image (165) of the fixator mounted thereon. The X-ray image 164 may correspond to the post-surgery lateral view 78 and the X-ray image 166 may correspond to the post-surgery lateral view 80 shown in FIG. 4. The digitized versions of these post-surgery X-ray images 164, 166 may be sent to the computer assisted orthopedic surgery planner software as denoted by block 76 in FIG. 3. Upon receipt of the post-surgery X-ray data, the computer assisted orthopedic surgery planner software module B 46 may act on the data to identify deviation, if any, between the suggested surgical plan data and the actual surgery data. Thereafter, module B 46 may revise the earlier specified distraction trajectory (at step 156 in FIG. 15) to assure a correct kinematic solution in view of any discrepancy between the pre-surgery plan data and the post-surgery data. Module B 46 may still optimize the distraction plan even if the fixator is not mounted exactly as pre-surgically planned.

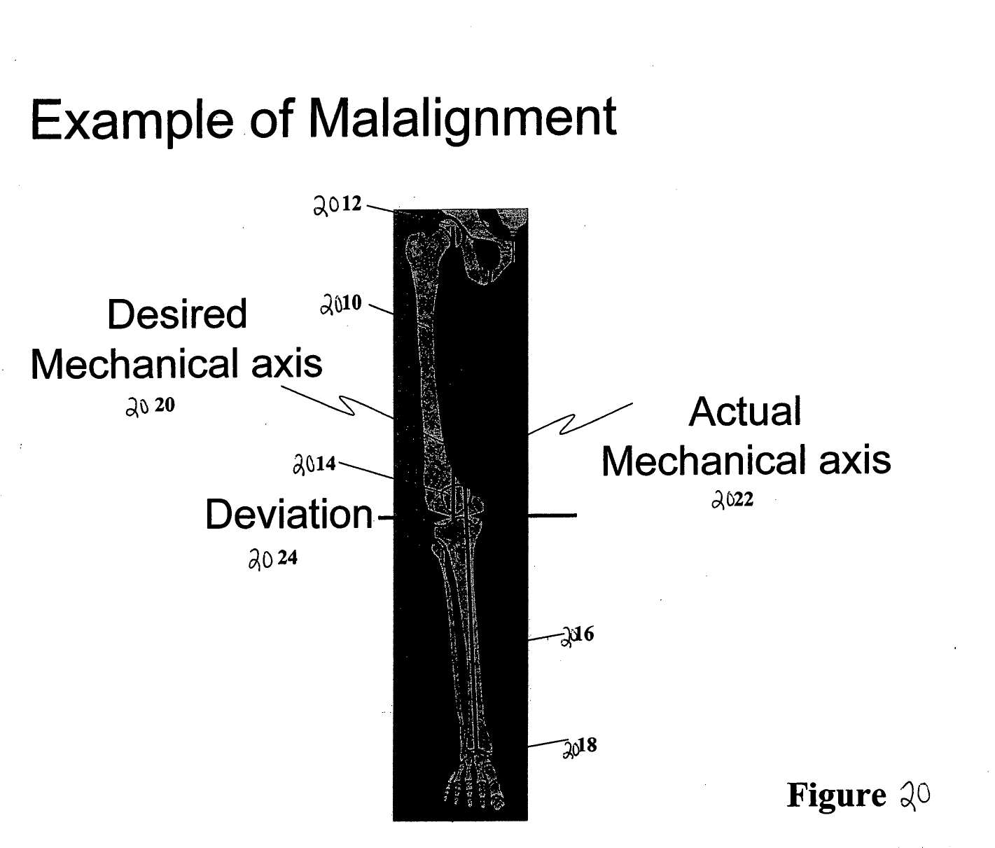

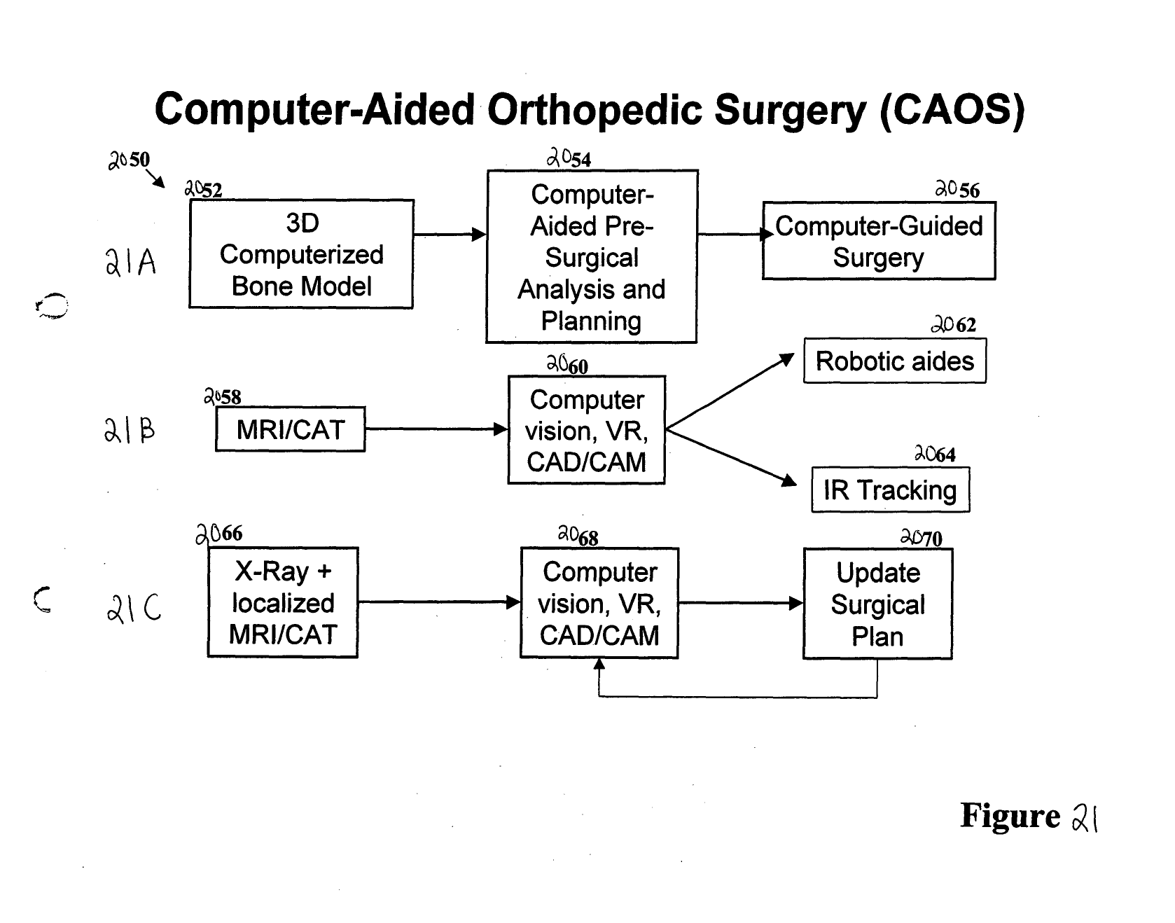

In one embodiment, to facilitate imaging and measurement of the fixator's position, a modified design for the fixator ring may be used. FIG. 19 illustrates an exemplary fixator ring 168 incorporating easily identifiable and detachable visual targets 170. The fixator ring 168 in FIG. 19 may be used as part of a ring for the Ilizarov fixator 20 (FIG. 1) or the Taylor Spatial Frame 73 (FIGS. 4 and 16). For example, the modified fixator ring 168 may replace the ring 24 in the Ilizarov fixator 20 shown in FIG. 1. The geometrical feature or targets 170 may be easily identifiable in computerized X-ray images. In the embodiment shown in FIG. 19, three posts (or targets or markers) 170 are attached to the ring 168 with each post having a unique geometry (here, the number of groves on the post) to identify the marker's 170 position in the X-ray image of the corresponding fixator. More or less than three posts may also be utilized. Furthermore, one or more posts may include a target sphere 172 at their open ends as shown. Thus, the surgeon may easily identify the fixator as well as the orientation of the fixator on the patient's bone.

After acquiring the X-ray image (e.g., a post-surgery X-ray image) and after performing automatic filtering, thresholding and edge detection on the X-ray image, the digitized X-ray image may be displayed on a window on a computer screen (e.g., the display screen 40 in FIG. 2 or a display screen of a computer at the remote site 32). The location of geometrical targets 170 may be done by a simple and reliable user-interactive mode. For example, the computer assisted orthopedic surgery planner computer 30 or the surgeon's computer at the remote site 32 may be configured to prompt the surgeon attending the computer to identify each target post 170 by moving the computer's cursor (or pointing with a computer mouse) over the approximate location of the marker's sphere 172 and then clicking to select. The computer may be configured (e.g., with a search software) to automatically search a bounded area to localize the sphere 172 and measure its relative position. This process may be done in both the AP and the lateral views. Similarly, the osteotomy/coricotomy may be located by prompting the surgeon to draw a line with the cursor (or with a computer mouse) over the osteotomy's location in the X-ray images. Because the position of each sphere 172 relative to the ring 168 that it is attached to would be known a priori, the positions and orientations of all rings on a fixator frame could thus be measured relative to the osteotomy/coricotomy. The targets 170, 172 could be removed from the fixator rings 168 before discharging the patient.

The foregoing describes exemplary embodiments of a computer assisted orthopedic surgery planner software according to the present invention. It is noted that although the discussion hereinabove focuses on the use of the computer assisted orthopedic surgery planner software for a patient's bone, the software may also be used for surgical planning and 3D modeling of any other anatomical part of the patient's body. Some of the major areas of applications of the computer assisted orthopedic surgery planner software of the present invention include: (1) Bone deformity correction including (i) osteotomy planning, simulation and assistance for, e.g., long bone deformities, complex foot deformities, (ii) acute fracture stabilization and secondary alignment in multiple trauma, and (iii) distraction osteogenesis case planning, simulation and assistance for, e.g., congenital and acquired deformities; (2) Maxillofacial as well as plastic reconstructive surgery; (3) Telemedicine or web-based surgical planning for physicians at distant locations; (4) Aide in the design of custom prosthetic implants; (5) Axial realignment when doing cartilage joint resurfacing; and (6) Creation of anatomical models for education of students and surgeons (e.g., for mock practice of surgical techniques).

The computer assisted orthopedic surgery planner software according to the present invention facilitates generation and simulation of accurate 3D models of a patient's anatomical part, e.g., a bone. Furthermore, in the complex area of bone distraction surgery, the computer assisted orthopedic surgery planner software makes accurate surgical plans based solely on a number of two-dimensional renderings or X-ray images of bone geometry. The software takes into account the complex and inherently three-dimensional nature of bone deformities as well as of fixator geometry when preparing a simulation of the proposed surgical plan prior to actual surgery. Complexities involved in accessing the target positions of the osteotomy and fixator pins surrounding the operated bone are substantially reduced with the help of CAD (computer aided design) tools and 3D simulation of surgical environment. Three-dimensional modeling allows for an accurate mounting of a fixator frame on the patient's bone according to a pre-surgical plan.

An Internet-based bone distraction planning service may be offered on a subscription-basis or on a per-surgery basis to surgeons located at remote places where computer assisted orthopedic surgery planner software may not be directly available. An expert surgeon may operate the service provider's computer assisted orthopedic surgery planner terminal to devise a surgical plan and distraction schedule for the remotely-located surgeon based on the X-ray image(s) data and other specific requests received from the remote surgeon over the Internet.

As noted hereinbefore, there are fewer than 1% of orthopedic surgeons who practice bone distraction. Furthermore, the external fixation with distraction currently takes an average of twelve to sixteen weeks at a cost of $1800 per week. However, even more time is required if the fixator was not initially properly mounted as often occurs in complicated cases. In these cases, the distraction schedule must be changed or the fixator must be reinstalled. The risk of major complications, including bone infection or fixation to bone failure rises exponentially when treatment times are extended. Complications and reinstallation of the fixator can require additional surgery costing $5000 to $10,000 and further extending the duration of fixation.

With the computer-aided pre-operative planning and frame application and adjustment methods described hereinabove, the duration of fixation (of a fixator frame) may be reduced by an average of four to six weeks. Additionally, by lowering the frequency of prolonged fixations, the cost savings may be approximately $9000 per patient. Shortening of the treatment time and reduction of complications may lead to better surgical results and higher patient satisfaction. The use of the computer assisted orthopedic surgery planner software of the present invention (e.g., in the Internet-based bone distraction surgery planning service) may make the frame fixation and bone distraction processes physician-friendly by simplifying fixation, decreasing preoperative planning time, and reducing the chances of complications through realistic 3D simulations and bone models.

The present invention broadly contemplates, in at least one preferred embodiment, a device and method for performing computer-aided surgery. The present invention may be specifically suited for performing computer-aided orthopedic surgery, such as an osteotomy, on misaligned bone. The following description provides an example of using the present invention to perform an open wedge osteotomy, but the invention can be used for many types of orthopedic and other surgeries.

Any reference to an open wedge osteotomy in particular is only by way of example.

FIG. 20 schematically shows an improperly aligned femur 10 and the resulting incorrect leg alignment. The FIG. 20 actual mechanical axis 2022 represents the axis of motion from the hip joint 2012 to the middle of the tibia 2018 (near the ankle). If correctly aligned, this axis 2012 should pass very close to the midpoint of the patella or kneecap 2014 (shown as the desired mechanical axis 2020). In the FIG. 20 example, there is a deviation 2024 between the desired 2020 and actual 2022 axes of motion. This deviation 2024 represents the amount of femur 2010 misalignment and can cause discomfort with decreased range of motion as well as other problems.

To correct these deformities, an orthopedic surgeon may perform an osteotomy or other surgery on the disfigured bone to return symmetry between these axes. Osteotomies are characterized by both the type of cut that is made in the bone (e.g., open wedge, closed wedge, center wedge) and the number of osteotomy sites (e.g., single, double). One type of osteotomy, an open wedge osteotomy, involves making a cut or wedge in the misaligned bone generally perpendicular to the long axis of the bone. Thereafter, depending on the desired bone realignment, the bone may be bent, twisted, and/or rotated about the cut sections until the “new” anatomical axis is properly aligned with the desired mechanical axis. Some type of fixation device, such as an internal plating system, may be used to hold the bone in its new orientation during the healing process after proper alignment is achieved, and a bone graft is used to fill in the open wedge to promote new bone growth.

As briefly described above, the movement necessary to realign a disfigured bone may be quite complex (movement around many different axes) and may require the solution of complex planning calculations as well as a certain amount of estimation based upon the experience of the orthopedic surgeon. To aid in the accuracy of this process, several types of Computer-Aided Orthopedic Surgery 2050 (CAOS) have recently been researched. In general, as seen in the flow chart of FIG. 21A, CAOS 2050 involves a three step process: (1) generating a 3D computerized model of the patient's bone 2052; (2) performing a computeraided pre-surgical analysis to aid in the creation of a surgical plan 2054; an (3) performing computer-aided surgery based on the pre-surgical plan 2056.

Traditionally, as shown in FIG. 21B, the 3D computerized bone model is generated from MRI or CAT data 2058 for the patient's bone. Use of the MRI or CAT data 2058 may produce an accurate 3D computer model of the bone, but these techniques are expensive and typically require an extended amount of time to perform the MRI/CAT procedure and to model the bone. Also, although generally available, the equipment necessary to perform these procedures may not be found in smaller hospitals or remote areas. Therefore, the use of these 3D modeling techniques, even when accurate, may require a patient to go through the time and expense of traveling to a different hospital.

Once a 3D computerized bone model is generated, computer vision, Virtual Reality (VR), Computer Aided Design/Computer Aided Manufacture (CAD/CAM), numerical optimization, artificial intelligence (AI), and/or other techniques and technologies 2060 may be used to help analyze the modeled bone and form a stepwise plan to carry out the surgery in the operating room. For example, a software program may compare the 3D model of the misaligned bone with existing models of properly aligned bones. The program may then determine, along a variety of different axes, an amount the bone needs to be moved in each direction Alternatively, the program may just analyze the actual and desired positions of the joints (e.g., hip, knee, and ankle) to aid in the determination of where to cut the bone for the osteotomy and how to reposition the bone.

The result of any of these procedures will preferably be a set of instructions or guidelines for the orthopedic surgeon to follow during surgery. The surgical plan may also calculate the positioning of one or more surgical tools or bone markers to be used during the procedure. Alternatively, the surgeon may be provided with a range (e.g., within 2 mm. of a certain position) of acceptable choices. The surgical plan will also preferably guide the surgeon in relocating or repositioning the misaligned bone. This part of the plan will preferably detail for the surgeon various distances and rotation angles through which the bone should be moved.

The surgical plan may be sent to the surgeon using various media types including: still images and illustrations; static CAD models and/or interactive CAD models; computer animations; video or movie presentations; text descriptions including cutting locations and angles any settings for surgical tools; rapid prototype models, or some other media type. The surgeon preferably reviews the plan and determines whether o not the surgeon is comfortable with performing the surgery according to the plan. If the plan is not acceptable, the surgeon preferably provides feedback and suggestions about the plan to aid in the development of a new plan. This process may repeat until the pre-surgical plan is acceptable to the surgeon.

After the surgical plan is reviewed, a computer-aided surgery may be performed by a variety of methods. For example, in FIG. 21B the surgery may be performed using robotic aides 2062 or some type of infrared (IR) tracking device 2064. One type of robot-aided surgery employs robots with touch sensors that register a patient's actual bone geometry during surgery. This actual geometry is compared with the 3D pre-surgical bone model to give feedback to the robot or surgeon while performing the surgery. This feedback allows a robot or surgeon to follow the surgical plan more accurately than without the sensors.

Alternatively, an IR marker system could be used during surgery. For example, IR markers may be attached to the patient's bone and to the surgical tools at various locations. A real-time IR sensing system may track these markers and register them to the pre-surgical 3D model to provide feedback to the surgeon or to guide the surgeon to make precise surgical cuts according to the pre-surgical plan. Again, this feedback allows the surgeon to more accurately follow the surgical plan.

As with the full MRI or CAT data modeling 2058, this real-time sensing and tracking of bone geometry using robotic aides 2062 and/or IR sensing systems 2064 is expensive, and only the most well-funded hospitals can afford the technology. Furthermore, many surgical procedures require computerized models of the entire limb (e.g., both the femur and the tibia of the leg) for generating the surgical plan, and acquiring MRI/CAT images of entire limbs may be both time-consuming and expensive.

Lower cost and more efficient and/or accessible surgical planning and performance methodologies are always desired. The present invention may improve upon conventional CAOS methods by replacing one or more of the above steps. For example, the MRI/CAT 3D-modeling step 2058 and the computer-guided surgical procedures 2062, 2064 may be replaced with more cost effective and/or quicker approaches. The entire CAOS process 2050 may be simplified and made more accessible for patient and surgeons by using less complex equipment and by locating certain computer equipment and planning resources in a centralized location.

The following example of the present invention describes using a planning computer and bone model database to generate a surgical plan for performing an orthopedic surgery.

In one aspect of the present invention, the 3D pre-surgical models of the misaligned bones may be created directly from readily available and inexpensive regular X-ray images 2066. Initially, a 3D model of a “normal” or properly aligned reference bone may be generated. This “template bone model” or template bone model CAD data may be generated based on representative bone topographies from MRI or CAT data, or data from any other imaging technique. The template bone mode may then be stored in a computer database for future access. The template bone model database preferably stores various different template bone models to be used for patients of different ages, genders, heights, and other characteristics. Alternatively or additionally, the bone models may be scalable or otherwise alterable to generate various-sized bone models. Once created and stored, each template bone model in this family of bone models can be used repeatedly and even shared among various surgeons, technicians, hospitals, or other interested users.

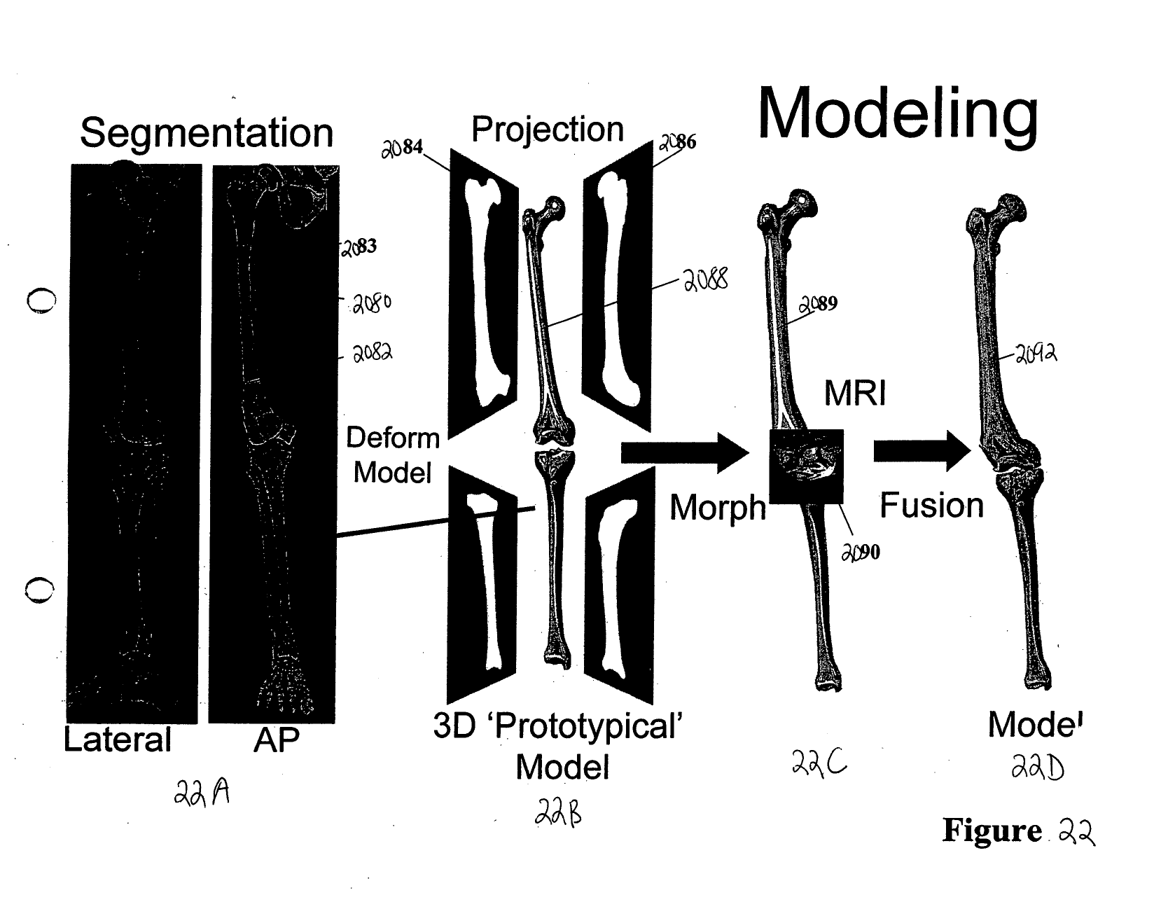

Preferably, each of these 3D template bone models 2088 can be graphically projected onto planes to produce a template bone model in a two-dimensional plane 2084, 2086 (see FIG. 22B). By projecting the 3D template bone model into at least two flat planes, preferably two planes that are orthogonal to each other, the template database or other computer can represent a bone as a series of two-dimensional pictures. An AP and lateral image projection of the template bone may be preferred. These two-dimensional projections may then be compared to X-rays or fluoroscopic images of a patient's bone to determine proper alignment. These template bones may exist in a computer database or other storage medium and can be shared, electronically or physically, with the users of several surgical planning computers.

The equipment used to generate the two-and three-dimensional template bone models is preferably a computer with advanced imaging and storage capabilities. The software algorithm is preferably able to convert the MRI, CAT, or other imaging data into a 3D “virtual” representation of the bone, as well as several flat projections of the bone. These parameters allow the template bone model to be reshaped or “morphed” to resemble the patient's actual bone. This modeling computer may exist separate from the planning computer (see below) and/or any other device, or the modeling and planning computers may be integrated into one unit.

Once the template bone model has been created, the surgeon or technician may prepare a 3D software “model” of the patient's misaligned bone. Rather than generating the 3D model of the improperly aligned bone directly from MRI or CAT data as performed by conventional systems, the surgeon or other technician may alternatively use several regular X-ray images of the patient 2066 (which are typically taken before any surgery). Preferably, at least a lateral and an AP (anterior-posterior) X-ray are taken of the patient's bone. The result of this imaging procedure is a series of two-dimensional representations of the patient's bone from various angles.

As shown in FIG. 22A, a software or other method may be used to segment 2080 the patient's bone 2082 in the X-ray images 2083. Segmentation is characterized by determining the outer bounds 2080 of the bones 2082 in the X-rays 2083. Segmentation may be accomplished using a light board and digitizing stylus. Because the AP, lateral and any other X-rays 2083 are preferably taken orthogonal to each other, the resulting segmented bone represents a projection of the patient's bone on orthogonal planes similar to the two-dimensional orthogonal planes of the template bone models just described.

A software program or other method may analyze the X-ray, of the patient's bone and compare it to the projections 2084, 2086 of the 3D template bone model 2088 (FIG. 22B). If the template database contains more than one set of template bone models, the software may select the template bone model that most closely matches the patient's bone. The selection of a template bone model may occur based on patient history, or the selection may be based on comparing the patient X-rays 2083 with two dimensional projections 2084, 2086 of the 3D template bone model 2088. The software then determines how the template bone model should be altered to more accurately depict the patient's actual misaligned bone.

A “morphine” software program may be used to alter, bend, or morph the selected template bone model 2088 in a way that causes the projections 2084, 2086 of the template bone model 2088 to more closely match the two-dimensional segmented bone images 2080 from the patient's X-rays 2083. In effect, the 3D template bone model 2088 is reshaped to resemble the patient's actual bone 2082. The result of this process is a computer-modeled 3D representation of the patient's bone 2089. The template bone model selection and morphing process may be performed on the modeling computer, the planning computer, a separate computer, or some combination of the three.

In a preferred embodiment of the present invention, the morphing software may alter the 3D template bone model 2088 in small iterations until the projections of the 3D bone model 2084, 2086 match the X-ray or other images 2083 of the patient's bone. For example, once an appropriate 3D template bone model 2088 is chosen, the software may analyze the differences between the two-dimensional projections of the 3D template bone model 2084, 2086 and the segmented images 2080 of the patient' bone. The software may then alter (stretch, bend, etc.) the 3D template bone model 2088 in such a way that the template bone model projections will more accurately resemble the patient's X-ray images. The projections of the altered template bone model may then be compared to the X-ray images again. If the projections and the X-rays are not yet sufficiently similar, the software preferably alters the 3D bone model again to achieve similarity. The newly altered bone model may then be projected and compared to the patient's X-ray images another time. This process preferably continues until the 3D bone model has been altered sufficiently to make the projections match the patient's X-rays. When sufficient similarity occurs, the altered 3D bone model (3D patient bone model 2089) should resemble the patient's actual bone.

This iterative reshaping may include a two-step process. For example, the positioning and scaling parameters may be optimized by rigid motion and scaling. An additional level of free-form deformation may be added for additional accuracy. As each iteration is completed, the 3D CAD data the defines the 3D patient bone model is preferably updated.

It should be noted here, that constructing a 3D patient bone model based on a pair of two dimensional X-rays will not typically result in a perfect representation of the patient's bone. The accuracy of the model is limited by geometric laws. In essence, the software algorithm described above codifies and improves upon the very method that a surgeon uses when looking at the same X-rays and then forming a mental picture of the patient's bone before surgery. The computer algorithm improves precision and makes this process easier for surgeons.

The “morphed” 3D patient bone model 2089, which now portrays the patient's bone, can be used to provide gross information about the alignment of the bone's mechanical and anatomical axes. In some cases, this bone model information may not be of sufficiently high fidelity or quality to accurately model the more geometrically complex areas of the bone, e.g., the joints at the ends of the bones. This gross information may also not properly show the ” twists, in the bone such as the relative orientation between the hip socket and the head of the femur In these cases, more accurate, local models of the joints or other bone areas can be derived from fusing selective volumetric MRI/CAT scan data 2090 for the joints with the morphed model 2089 (see FIGS. 22C-22D). Portions of the 3D patient bone model may be reconstructed or refined using selected MRI cross-sectional slices of the patient's bone 2090, or portions of the bone model may be completely replaced with the MRI data. FIG. 22C shows a “local” MRI 2090 taken at the bottom of the femur to augment and clarify the morphed bone model 2089 at an area of bone surface complexity. However, if the localized MRI information is not available, the morphed bone can still be used with the present LAOS invention as an improvement over current methods.

The result of this process is preferably a 3D software model 2092 (based on 3D CAD data) of the patient's bone that is sufficient for computer-aided planning of the orthopedic surgery or other procedure In contrast to conventional methods, this model 2092 is preferably created using normal patient X-rays 2083 and pre-existing template bone models 2088 that may be generated once and then shared among various users at different imaging locations. This method may decrease the amount of time and money spent generating the 3D software model 2092 of the patient's misaligned bone.

In addition to the 3D patient bone model 2092 created on the computer, a rapid prototype model of the bone could be created using the stored 3D CAD data. The rapid prototype model is an actual, physical model of the bone made using conventional CAD/CAM or other modeling techniques. This rapid prototype model may be given to the surgeon to allow the surgeon to better visualize the misaligned patient bone before and even during surgery.

After the morphed 3D patient bone model 2092 is generated, CADS planner software developed as part of the present invention, may initially determine the osteotomy site location on the model. Alternatively, the surgeon may draw on his experience to choose a location for the osteotomy, and the location may be further optimized by the CAOS planning software. In some osteotomy procedures, the patient's bone ma be so poorly aligned that a multiple osteotomy is needed to restore alignment to the bone. In these multiple osteotomies, the planning computer may be especially useful because of the complexities of the 3D model.

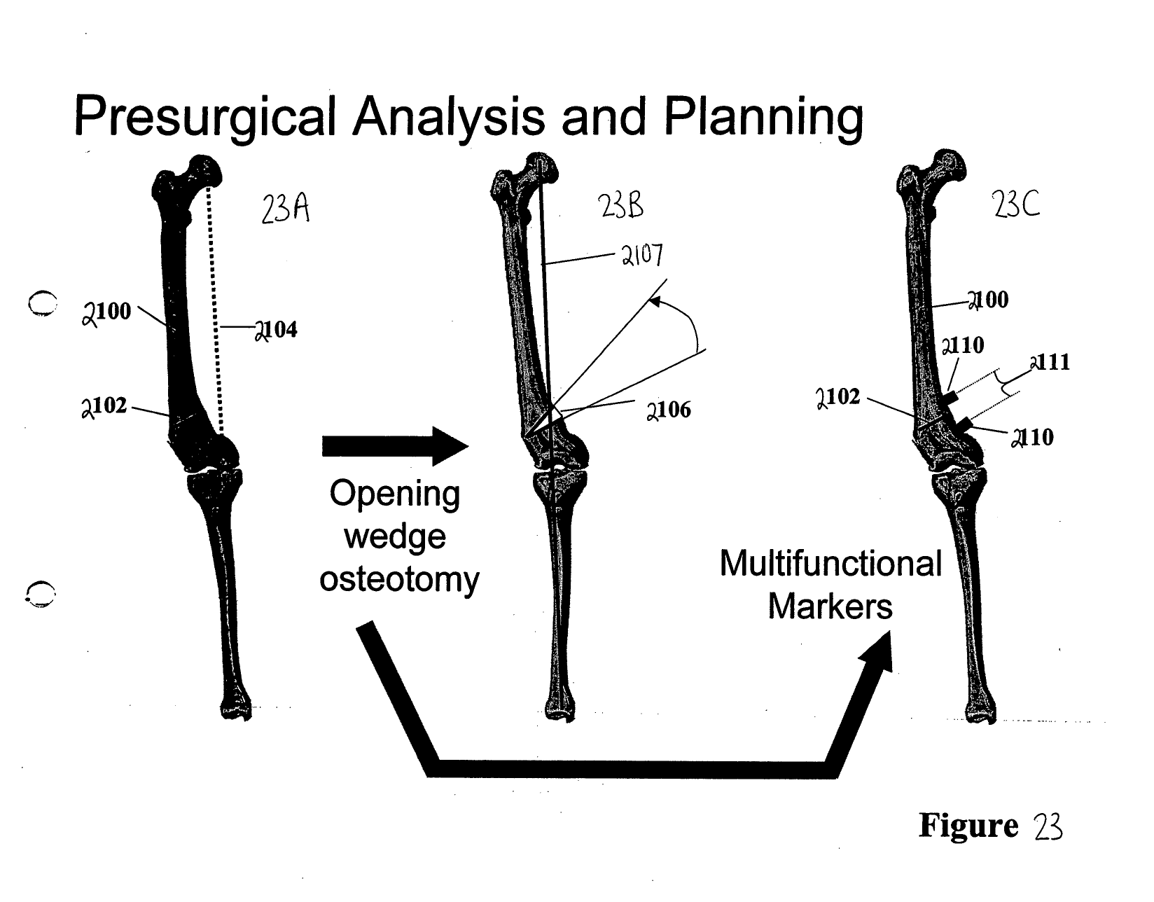

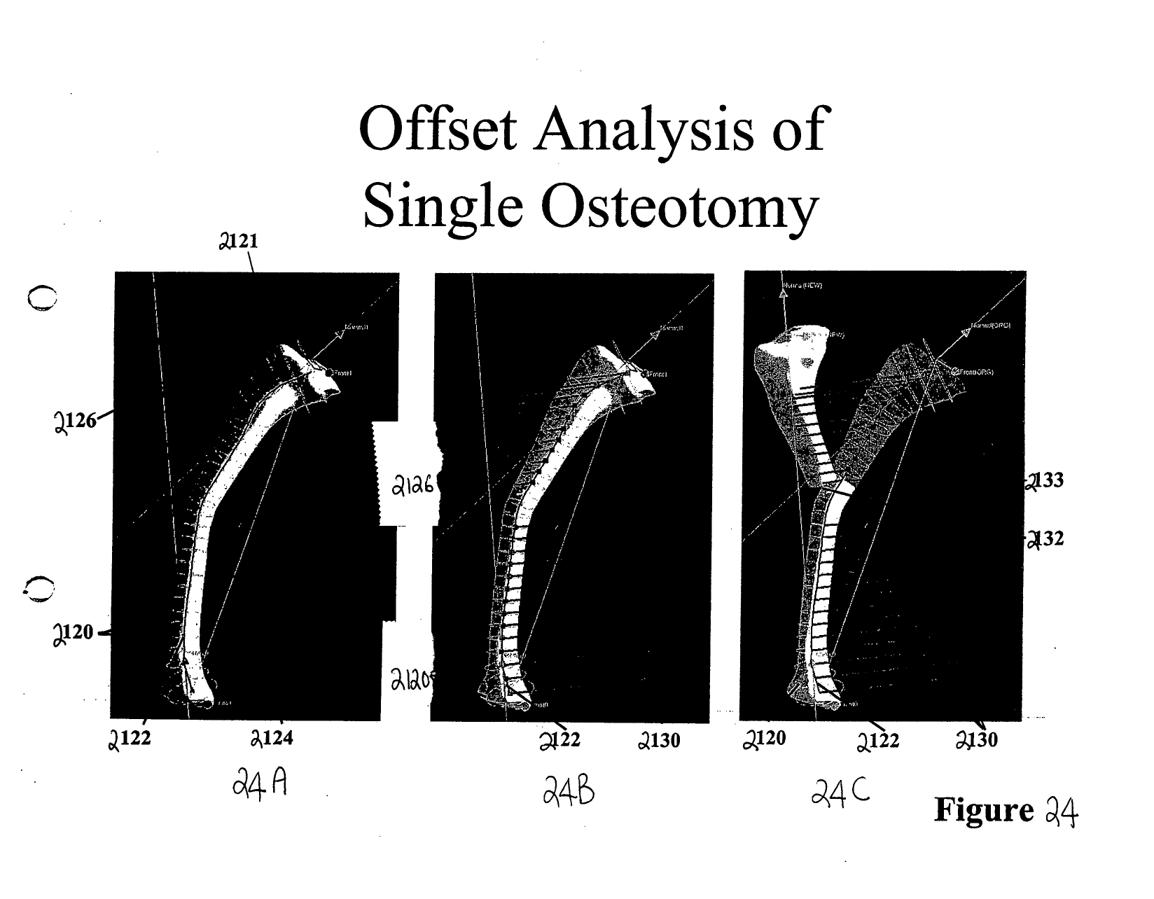

The CAOS system preferably includes a planning computer that may or may not include the database of template bone models 2088, the computer that modeled the original template bone models 2088, or the computer that morphed the template bone model 2088 to create the patient bone model 2089. FIGS. 23A and 23B show a rudimentary determination of the proper osteotomy procedure. In FIG. 23A, the planning computer software analyzes the alignment of the leg bones 2100 and the present mechanical axis of motion 2104 (the dotted line from the ball joint in the hip to the bottom of the tibia in the ankle). FIG. 23A depicts the software' determination of a cutting location 2102, and FIG. 23B depicts an opening “wedge” angle 2106 as a possible solution that realigns the mechanical axis 2107 through the middle of the patella.

There may be different software algorithms used for the various types of orthopedic surgeries. For a “simple” single osteotomy, the software algorithm may utilize the steps as set forth in FIG. 24. The single osteotomy entails locating an optimum place to cut the bone which limits the amount of bone movement needed to realign the bone during surgery. FIG. 24A shows a 3D bone model 2121 of a patient's misaligned bone. The planning software may initially determine an anatomical axis 2122 through the center of the bone model 2121. The computer may also calculated an existing mechanical axis 2124 defined from the midpoint at one end of the bone down to the midpoint of the other end of the bone. The software algorithm preferably also calculates desired mechanical axis 2126 that will extend between the midpoints of the two ends of the bone after the osteotomy is performed. This desired mechanical axis 2126 should begin at the existing midpoint of one end of the bone (shown extending from the lower end of the bone in FIG. 24A), and extend along the intended orientation of the bone.

The objective of the planning software is to determine at what location to cut the bone so that the relocation of the bone from its present mechanical axis 2124 to the desired mechanical axis 2126 is at a minimum. To accomplish this task, the anatomical axis 2122 of the bone is preferably sliced or segmented at regular intervals 2120 throughout the 3D model. These slices 2120 are preferably taken perpendicular to the anatomical axis 2122 of the bone 2121. In the FIG. 24A example, there are 20 slices 2120 taken.

The planning computer then preferably “virtually” cuts the bone model 2121 at each of these 20 slice locations 2120 and moves the upper section of the bone until the midpoint of the upper end of the bone is aligned with the desired mechanical axis 2126 (FIGS. 24B-24 C). The planning software may then compare the “new” midpoint (anatomical axis) of the bone section just above the bone slice 2132 with the position of this same point before the relocation 2133. The distance between these two points 2132, 2133 is the deviation that now exists between the upper and lower bone segments in FIG. 24C. The planning computer preferably calculates this deviation distance for each of the 20 (or any number) of slice 2120 iterations and determines which osteotomy location has the smallest deviation (all deviations shown as 2130). This location is preferably chosen as the preliminary site of the osteotomy.

A more complicated software methodology may be employed to perform the predictive analysis of the osteotomy. For example, a “rough” analysis to determine general location could then be followed up with a more refined analysis in the general vicinity of the predicted osteotomy location. Also, additional slices could be used for better resolution.

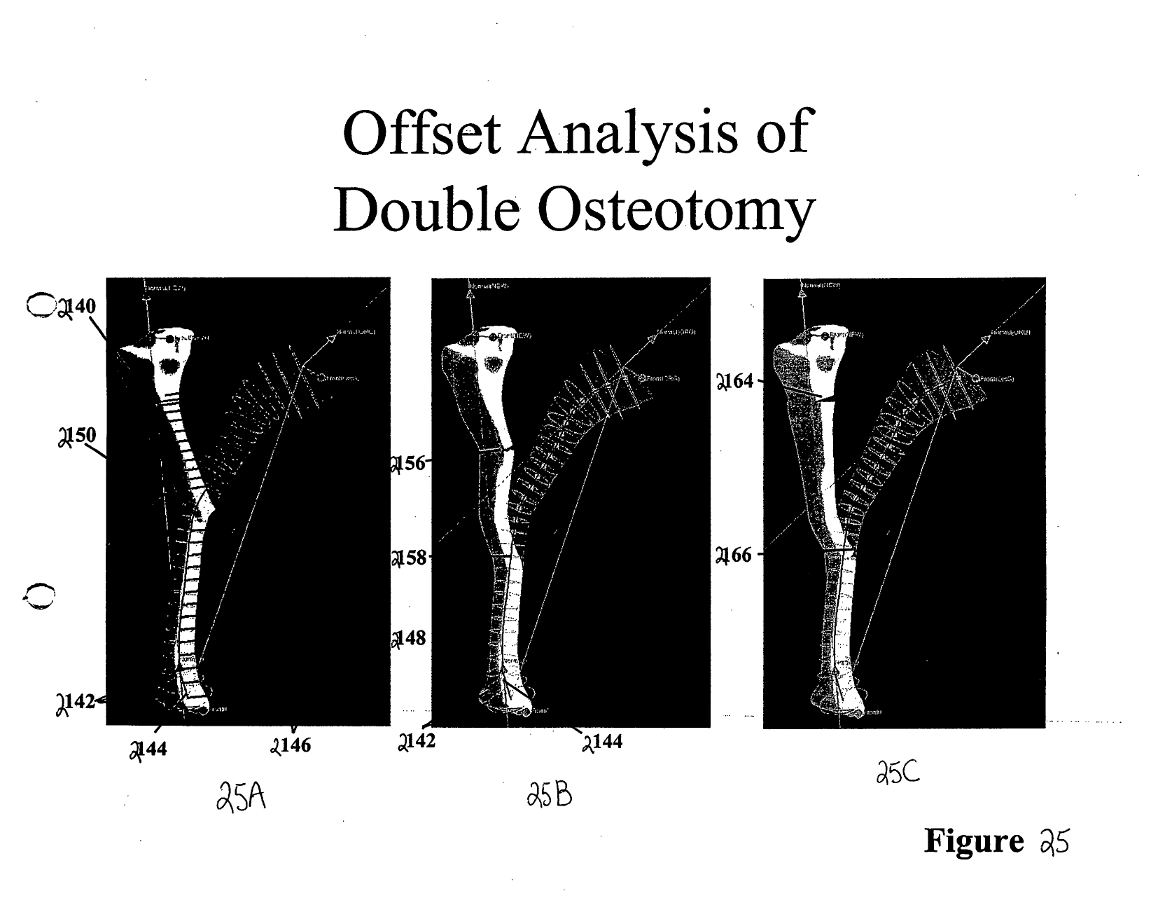

FIG. 25 shows a possible software methodology for use with E double or multiple cut osteotomy planning procedure. The axes 2144, 2148, 2150 and slice locations 2142 in this method are preferably determine in th, same way as the single osteotomy. However, the planning software may now perform a more complicated predictive analysis.

FIG. 25A shows that the same 20 virtual slices 2142 are taker as in the single osteotomy procedure. However, the planning software preferably goes through all possible iterations of osteotomy locations. With a single osteotomy and 20 slices 2142, there are 20 iterations. With double osteotomy and 20 slices, there are just under 2200 unique iteration (discounting the iterations that would duplicate the same osteotomy locations but in a different order).

For example, the planning software may start from the first slice location and “perform” a first virtual osteotomy (FIG. 25A). Thereafter, the planning software may calculate a second osteotomy performed from each of the other 19 slice locations. To calculate the effect of the osteotomy, the planning software preferably adds the deviation of the anatomical axes for both osteotomies together.

After these 19 (or any number) iterations have been modeled and the deviation results have been calculated, the planning computer preferably moves the first osteotomy location to the second slice and continues the analysis. The second osteotomy is preferably “made” at the other 19 slice locations, and the deviation results of the two cuts are preferably added to determine a total deviation.

After all 20 of the slice locations have been modeled with the other 19 slice locations for a second cut, the planning software preferably plots the results on a 3D diagram. For example, the X and Y axes could represent the first and second slice locations, and the Z axis could plot the total deviation at these two osteotomy locations. By examining the diagram, and looking for a Z axis minimum, the planning computer or surgeon may easily determine the appropriate locations for the double osteotomy or other multiple orthopedic procedures.

Although a surgeon may be able to visualize in his or her head the appropriate location for a single osteotomy, a double or higher order osteotomy, as shown in FIGS. 25B-25C, may be too complicated for such human analysis. In these cases, the CAOS method of the present invention may be especially useful.

After determining the proper procedure location, the computer-based planning software places multifunctional markers 2110 near the suggested osteotomy location 2102 on the computerized 3D patient bone model 2100 (see, FIG. 23C). The multifunctional markers 2110 may be used to both register bone location during surgery and anchor various surgical guides (e.g., a cutting guide to open the bone, a fixation guide to reposition the bone in the desired orientation during surgery, or a combined cutting-fixation guide). The optimizer or planning software ma determine the appropriate location for the markers 2110 based on mechanical tolerance data for the surgical guides that will be used during the surgery. Preferably, the guides and markers 2110 have already been modeled by the planning computer. These multifunctional markers 2110 may be detected during the surgery by X-ray, fluoroscopy, or other imaging methods to increase procedure accuracy over conventional surgical methods. The computer may provide an exact preferable location in which to place the markers 2110, or the computer may offer a suggested range of marker positions (an allowable work envelope) within an acceptable tolerance limit.

Based on the computer-aided marker placement position, the planning computer preferably generates a “preliminary” surgical plan for the surgeon to follow in the operating room. This surgical plan may help the orthopedic surgeon decide whether or not to perform the surgery, any the plan may be used in the event the final surgical plan (explained below) is lost or electronically unavailable during surgery. This preliminary surgical plan preferably includes step-by-step guidelines for performing the orthopedic surgery. For example, the surgical plan may include various translation and rotation settings for an adjustable cutting guide used to locate and hold a reciprocating saw during surgery. Because the planning computer has previously calculated the location of the markers, and further because the adjustable cutting guide is anchored to the multifunctional markers during surgery, the cutting guide “settings” can be pre-calculated as part of the preliminary surgical plan. During the actual surgery, the surgeon need only set the cutting guide according to the plan and attach the guide to the markers (or attach the guide to the markers and then adjust the guide settings).

The planning computer may also calculate a pre-surgical plan for an adjustable fixation guide. This guide, which is preferably used to open the osteotomy wedge and reposition the incorrectly aligned bone, may also be anchored to the multifunctional markers. Because the marker position has previously been determined, the planning computer can also predetermine the fixation guide settings.

In an alternative embodiment, the cutting, fixation, or combined cutting-fixation guides may attach directly to the bone without the use of multifunctional markers. Preferably, these guides will be adapted for direct mounting to the bone using an adhesive, screw, or other device. Moreover, as described below, the present invention may be used when these guides are attached without the use of a pre-surgical plan, for example after a trauma. Images may be taken of the bone with attached guides, and a “final” surgical plan could be developed without the presurgical plan.

Once a planning computer has either modeled or simulated the osteotomy procedure, or has developed a detailed preliminary surgical plan, the simulation and/or plan is preferably sent to the surgeon to determine if the procedure will be performed. The surgical plan may be sent to the surgeon using various media types including: still images and illustrations; static CAD models and/or interactive CAD models; compute animations, video or movie presentations; text descriptions including cutting locations and angles and settings for surgical tools; rapid prototype models, or some other media type. The surgeon can preferably view the 3D computer simulation or other plan of the surgery and decide whether or not the plan is acceptable. If the surgeon does not “accept” the plan in its current embodiment, the surgeon may provide suggestions or comments that are sent back to the planning computer operator or to the surgical expert counseling the planning computer operator. The simulation and acceptance of the surgery may occur before a detailed preliminary surgical plan is developed, or the plan may be presented to the surgeon so they can proceed with the proposed plan or offer a new plan.

Once the patient's bone has been properly modeled and/or a preliminary surgical plan has been developed, the patient is ready to undergo the actual orthopedic surgical procedure. During surgery, radio opaque multifunctional markers 2110 are preferably attached to the patient's bone as both a location mechanism and as an anchor for the surgical guides (e.g., a cutting guide, fixation guide, combined cutting-fixation guide, and/or a calibration grid), These markers 2110 may be small blocks that include a screw for mounting the markers to the bone and a screw acceptor (threaded hole) for attaching one or more guides thereto. Alternatively, the markers may be a threaded pin that is inserted into the patient's bone. Various surgical tools could be clamped to the end of the pin extending out of the bone during surgery.

At the beginning of the surgery, the surgeon is shown a CAI display or other representation of the pre-surgical plan depicting the required location/orientation of the osteotomy and the ideal location (and/or a tolerance zone) for the markers 2110 to be placed on the patient' bone. The surgeon then exposes the patient's bone at the general location of the intended osteotomy and manually inserts the markers onto the bone (e.g., by screwing the markers into the bone) in approximate locations above and below the intended osteotomy. Because of the inherent inaccuracies associated with a surgeon trying to duplicate the location seen on a “picture” of the bone, the markers may or may not be placed in the exact desired location. Because of the accuracy of the plan, the surgeon may generally perform a minimally invasive surgery using a smaller incision than conventional methods.

In attaching the multifunctional markers to the patient's bone, it is preferable to align the axes that extend vertically through the top of the two markers (axes 2111 in FIG. 23C) so that these axes are parallel to each other. If these axes are parallel, the markers 2110 are “in the same plane” which will make mounting the various guides to the markers easier. This may also make the guides simpler to design as fewer degrees of freedom for placement are needed. To accomplish this alignment, a marker insertion guide (not shown) may be used to align the markers during positioning on the patient's bone.

The marker insertion guide is preferably a hollow metal tube with jagged edges towards one end. The jagged edges can “grab” the bon and secure the hollow tube while a drill bit is extended through the tube. The tube acts as a guide to make sure the markers are attached in the same plane. Various types of marker insertion guides are well-known in the medical arts.

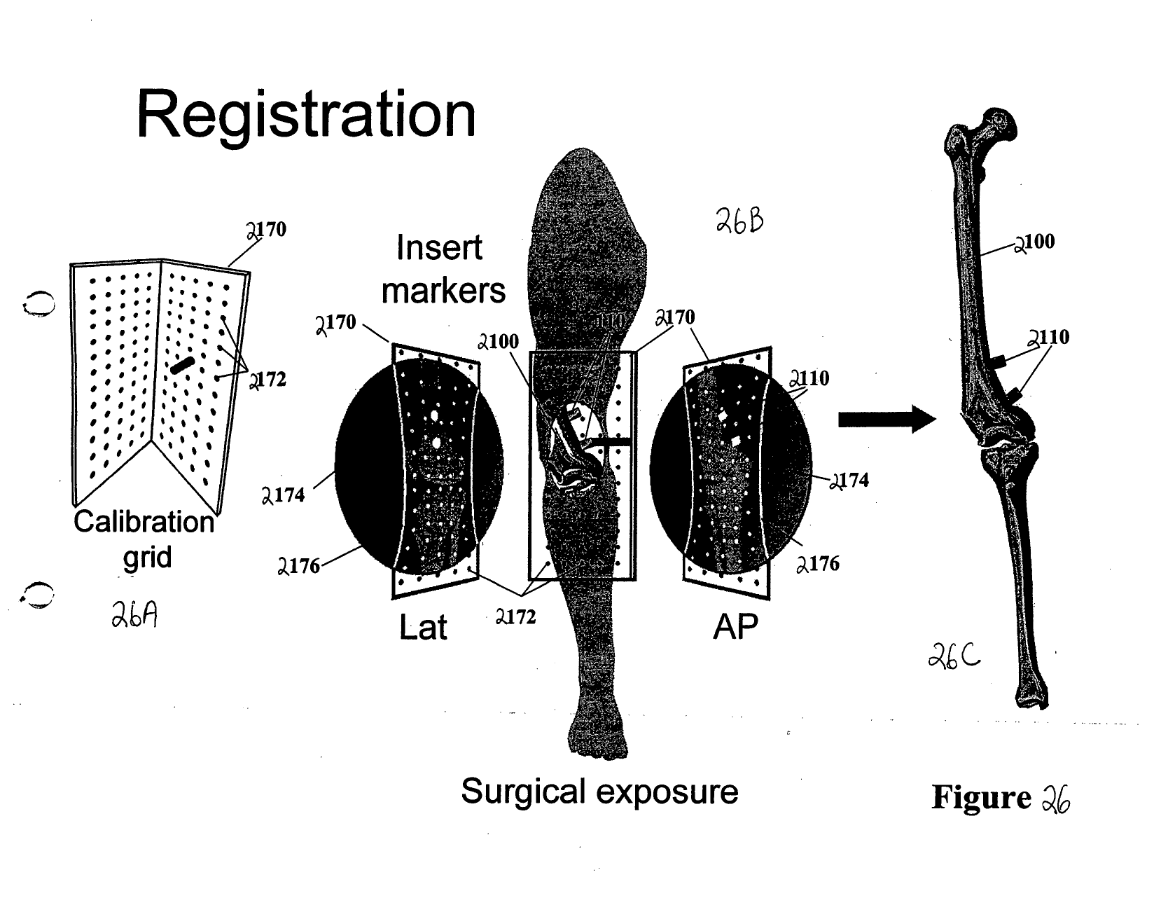

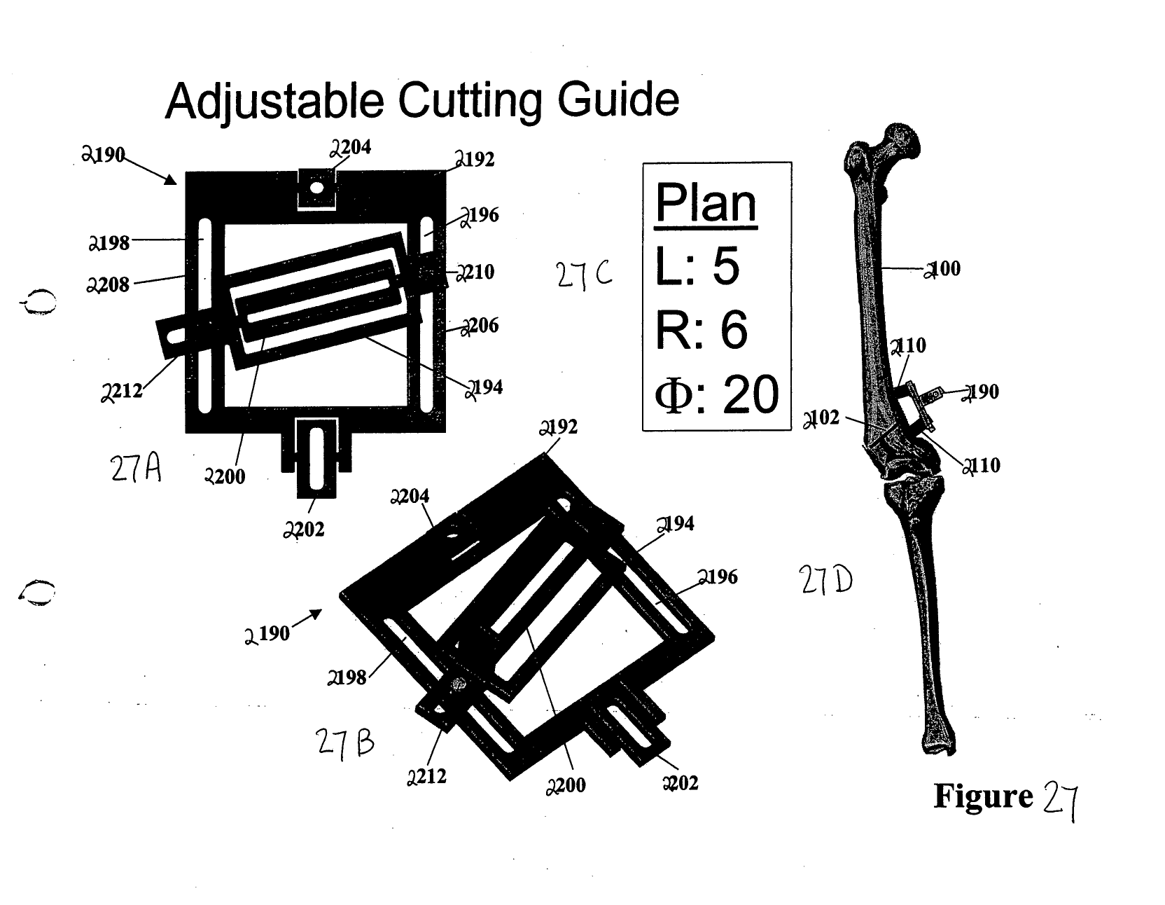

After the markers 2110 are attached, a translucent calibration grid 2170 (with radio-opaque grid points or gridlines 2172 printed thereon) may be mounted around the patient's bone (FIG. 26A). The calibration grid 2172 preferably consists of two orthogonal grid sheets that are mounted around the outside of the surgical area such that they are parallel to the image plane of a lateral and AP fluoroscopic image of the misaligned bone. For example, the grid 2170 may be mounted to the multifunctional markers 2110. Upon imaging, the two-dimensional planar image 2174 of the bone is set against the backdrop of the grid points 2172. These grid points 2172 are used to more accurately determine the positioning of the markers 2110 and other areas of the bone by providing background reference to aid in the “unwarping” of the fluoroscopic image

After the calibration grid 2170 is secured in place, one or more fluoroscopic images 2174 are obtained for the exposed bone area including the attached calibration grid 2170. Preferably, at least a lateral fluoroscopic image and an AP fluoroscopic image are obtained (FIG. 26B). A fluoroscopy is a low radiation imaging device that can be more flexible and useful in certain situations than obtaining X-ray images. A fluoroscopy machine is generally more maneuverable as compared to the bulkier and more cumbersome X-ray machine. However, fluoroscopy is often susceptible to image warping effects (see, e.g., 2176) caused by surrounding magnetic or electromagnetic fields, sagging of the imaging source or other interference. The warping 2176 of a fluoroscopic image of an object distorts the image. Therefore, image translation and further “unwarping” may be performed to remove or minimize resulting distortion 2176. This type of fluoroscopic image correction is well-known i the art and is typically corrected using software techniques. The “warped” calibration grid points in the fluoroscopy can be used to unwarp the fluoroscopy image. When the imaged grid points are straight, the image has been unwarped correctly.

The corrected fluoroscopic image is generated on or sent to the planning computer, to determine the location of the markers 2110 as precisely as possible in relationship to the 3D bone model. (FIG. 26D). The “new” or updated multifunctional marker position analysis may help negate the inherent problems with actual marker positioning (e.g., not being able to accurately place the markers according to the plan). The planning computer software preferably updates the locations of the earlier placed markers 2110 on the 3D patient bone model 2100 to reflect the actual marker locations on the patient's bone. With this updated information, the planning software may then re-calculate the pre-surgical plan setting for the cutting guide, fixation guide, combined cutting-fixation guide, and any other device used during the surgery. In essence, the pre-surgical plan may be updated to correct the inherent errors in placing the marker by hand during surgery. Using this intra-operative feedback during the surgery, a more accurate surgical plan can be calculated by the planning computer.

After the new or “final” surgical plan is calculated, the surgeon is ready to actually perform the osteotomy. The osteotomy preferably begins by cutting the bone so that the bone can be repositioned according to the desired axis of motion. The bone is typically cut using a reciprocating or “gigley” hand-held saw that is less damaging to surrounding tissues and cells. To more accurately control the cut made 1 the reciprocating saw, an adjustable cutting guide 2190, such as the one shown in FIG. 27A, may be used.

FIG. 27A details a top view and FIG. 27B shows an isometric view of a manually adjustable cutting guide 2190 that may be mounted onto the multifunctional markers 2110 secured to the patient's bone 2100. The cutting guide 2190 is comprised of a base plate 2192 and a cutting guide member 2194. The base plate 2192 preferably has two anchor slots 2202, 2204 through which a screw or other attachment device may be inserted so that the adjustable cutting guide 2190 can be secured to the multifunctional markers 2110 on the patient's bone 2100. The base plate 2192 may also include two adjusting slots 2196, 2198 for setting the proper positioning of the cutting guide member 2194. The two adjusting slots 2196, 2198 are preferably marked with indicators 2206, 2208, in this case numbers, that correspond to the surgical plan that the planning computer outputs for the surgeon.

The cutting guide member 2190 includes two screws 2210, 2212 or other adjusting devices that are integrally located within the adjusting slots 2196, 2198. In the center of the cutting guide member 2194, there is preferably a saw slot 2200 that can be rotatably adjusted to accommodate reciprocating saw at a variety of angles for cutting the patient's bone. In practice, the adjustable cutting guide 2190 may allow the surgeon to more accurately recreate a cut in the patient's bone as modeled by the planning computer. Preferably, the surgical plan calculated by the planning computer, after being updated to reflect the actual positioning c the radio-opaque multifunctional markers 2110 on the patient's bone, includes a preferred “setting” for the adjustable slots 2196, 2198, as well as a preferred angle Φ for the saw slot 2200. One such example plan is show in FIG. 27C.

The slot settings 2206, 2208 for the adjustable cutting guide 2190 are preferably used to locate, along at least two axes, the reciprocating saw used to cut the patient's bone. For example, by altering the relative position of the slot settings with respect to each other, the cutting guide member 2194 may be rotated in a plane perpendicular to the face of the patient's bone (looking down on the bone from above). If the right slot setting 2206 is set to 5, and the left setting 2208 is moved from 1 up through 12, the cutting guide member 2194 will rotate in a clockwise direction. Because the left set screw 2212 of the cutting guide member 2194 preferably has a slotted opening rather than a simple hole (as on the right side 2210 }, the cutting member 2194 is preferably capable of being rotated. If both the left and right set screws 2210, 2212 of the cutting guide member were in circular holes, the cutting guide member 2194 would only be able to slide back and forth in the base member adjustable slots 2196, 2198.

In addition to setting the vertical rotation of the cutting guide member 2194, the slot settings 2206, 2208 also determine at what location relative to the multifunctional markers 2110 the cut should be made. For example, if both slot settings 2206, 2208 are increased by the same amount, the cutting guide member 2194 will slide up towards the to. marker while maintaining the same rotational setting. Likewise, if the slot settings 2206, 2208 are decreased in equal amounts, the cutting guide member 194 will move towards the lower marker. Both slot settings 2206, 2208 are preferably manipulated and set via manual setting devices such as a set screw 2210, 2212 or small bolt used to tighten the cutting guide member 2194 in the desired position. Because the surgical plan preferably displays the appropriate guide settings to the surgeon and the adjustable cutting guide slots are pre-marked, use of the cutting guide may be quicker, easier, and more accurate than conventional methods.

The surgical plan also preferably includes an angle Φ for which the saw slot 2200 is to be rotated and secured within the center of the cutting guide member 2194. As with the base plate slots 2206, 2208, the saw slot 2200 is preferably pre-marked with angle demarcations (not shown) that allow for easy adjustment of the saw slot 2200 to a desired cutting plane angle. The saw slot 2200 may then be secured in a desired position by way of a set screw or some other temporary fixation device.

Once the three (or more) settings for the adjustable cutting guide 2190 are set according to the updated surgical plan, the cutting guide 2190 is preferably attached to the patient's bone 2100 (or the guide 2190 may be attached to the bone 2100 before setting). Preferably, the base plate 2142 of the adjustable cutting guide 2190 includes two mounting slots 2202, 2204 through which a screw or other mounting device can be inserted to affix the adjustable cutting guide 2190 to the multifunctional markers 2110. The first mounting slot 2204 is preferably a hole slightly larger than the mounting screw so that the cutting guide 2190 is unable to slide with respect to the markers 2110 during surgery. The second mounting slot 2202 is preferably oval or slotted to accommodate a slight “misplacement” of the multifunctional markers 2110 on the patient's bone 2100. Because the markers 2110 may not be placed at exactly the desired distance apart from each other, the mounting slots 2202, 2204 can preferably accommodate the markers 2110 at slightly greater or smaller distances from each other. FIG. 27D shows the adjustable cutting guide 2190 mounted to the multifunctional markers 2110 on an exposed bone 2100 with the skin and other tissues removed for clarity.

After the adjustable cutting guide 2190 is mounted to the bone 2100, the surgeon preferably inserts a reciprocating saw or other cutting device into the saw slot 2200 of the cutting guide 2190 and cuts the patient' bone 2100 according to the surgical plan. The saw slot 2200 may include a mechanical stop that prevents the saw from cutting a slot in the bone of more than the desired depth. After the osteotomy cut is made, the saw is removed from the cutting guide 2190 and the cutting guide is dismounted from the bone 2100 by unscrewing it from the multifunctional markers 2111.

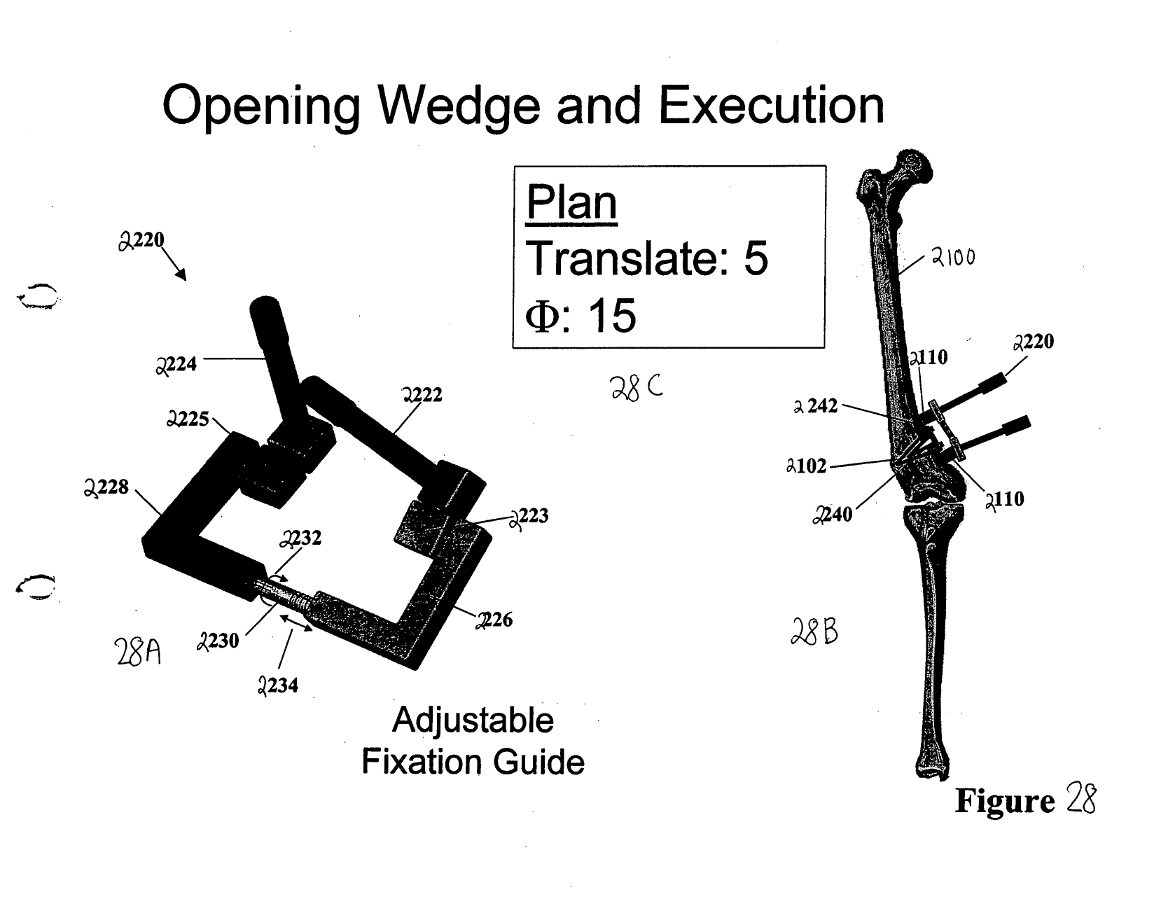

After the cutting guide 2190 is removed from the bone 2100, and with the multifunctional markers 2110 still attached to the bone, the bone is ready to be bent, rotated, twisted, and/or repositioned into the proper alignment according to the updated surgical plan. The cut 2102 in the bone 2100 has been made, and the wedge may now be opened. FIG. 28 shows an exemplary adjustable fixation guide 2220 for use in repositioning an improperly aligned bone. The purpose of the adjustable fixation guide 2220 is preferably to force the bone 2100 into the newly desired position with a greater amount of accuracy compared to conventional methods. Again, this part of the surgical plan has been “updated” based on the actual position of the multifunctional markers.

The adjustable fixation guide 2220 pictured in FIG. 28A allow for movement of the bone along two axes: (1) lengthening the space between the two markers 2234 and (2) rotating the two markers away from each other 2232. The fixation guide 2220 is preferably made of two guide arms 2226, 2228, two mounting tabs 2223, 2225, two base arms 2222, 2224, and a base shaft 2230. By manipulating the two guide arms 2222, 2224 according to the surgical plan, the osteotomy wedge may be opened precisely according to the computer-calculated optimum position based on the updated location of the multifunctional markers 2110.

The two base arms 2226, 2228 are preferably connected to each other by a base shaft 2230 that runs at least partially into and through the middle of the base arms 2226, 2228. The base shaft 2230 allows the base arms 2226, 2228 to move translationally 2230 (towards and away from each other down the long axis of the base shaft 2230) as well as rotationally 2232 (around the long axis of the base shaft 2230). At the opposite ends of the base arms 2226, 2228 from the base shaft 2230, there are preferably two mounting tabs 2223, 2225 and two guide arms 2222, 2224. The mounting tabs 2223, 2225 provide a surface for securing the adjustable fixation guide 2220 to the multifunctional markers 2110. For example, the mounting tabs 2223, 2225 may have a post or threaded shaft extending out from the bottom of the adjustable fixation guide 2220 that can be inserted into the multifunctional markers 2110.

FIG. 28B shows the adjustable fixation guide 2220 mounted on the multifunctional markers 2110. FIG. 28C shows an exemplary surgical plan for manipulating the two guide arms 2222, 2224 in order to open the wedge 2102 in the osteotomy. In this example, the translation is set to 5 and the rotation Φ is set to 15. These numbers can represent degrees, millimeters, are any other dimension, or may just represent position numbers labeled on the adjustable fixation guide 2220. In any case, the surgical plan enables the surgeon to accurately manipulate the guide arms 2222, 2224 of the adjustable fixation guide 2220 to open the bone wedge 2102 or otherwise relocate the bone 2100. The guide arms 2222, 2224 may be ratcheted to prevent the bone from closing if pressure is removed from the adjustable fixation guide 2220.

FIG. 28B also shows a fixation plate 2240 that may be used to hold the opened wedge 2102 in the appropriate position while the bone 2100 heals and rebuilds itself. The plate 2240 may be a metal rectangle with two small holes drilled therethrough near the ends of the fixation plate 2240. Preferably, while the adjustable fixation guide 2220 is still connected to the multifunctional markers 2110, the fixation plate 2240 is secured to the open wedge-side of the bone 2100. To aid in the healing process and to promote future bone growth, bone material from a bone graft may be inserted into the wedge 2102 to fill in the empty space.

As stated above, it should be noted at this point that the above cutting and fixation guides are presented by way of example only. In practicing the present invention, these two guides may be combined into one cutting-fixation guide, or any number of other surgical tools or guides may be used. Likewise, the various surgical tools and/or calibration guides could be attached directly to the bone, without the use of the multifunctional markers. In this embodiment, a fluoroscopic image of the attached tool could be captured during surgery to update the surgical plan. A number of different variations on these same themes, including methods without a pre-surgical plan, could be employed within the scope of the present invention.

If the osteotomy procedure of this example includes more than one cut, the other parts of the bone may be opened at this time. As with the first procedure, the markers are placed; a fluoroscopic image is taken; a final surgical plan is developed; and the bone is cut, opened, and realigned. To save time, the marker placement and fluoroscopy for both sets of cuts may be completed at the same time. Once the final surgical plan is generated, each cut may then proceed in turn.

After the open bone wedge 2102 is filled and the fixation plat 2240 is secured, the adjustable fixation guide 2220 is removed from the multifunctional markers 2110. The markers 2110 themselves are preferably removed from the patient at this point. However, in some applications of the present invention the markers may be necessary for a future surgery or adjustment and are not removed from the bone immediately after surgery. Specialized markers (not shown) may be needed if the markers are not removed. After removal, the surgical area is closed and the surgery completed. During recovery, additional X-rays or other images may be taken to determine if the osteotomy was performed successfully.

The above example described an embodiment of the present invention wherein the modeling computer, planning computer, and all necessary surgical equipment exist in the same location where the surgery is performed. A computer network, such as the Internet, may also be use to connect the operating room equipment to the planning and other computer systems. In this way, one central planning computer location can serve a plurality of different operating rooms or different hospitals. Alternatively, one central modeling computer may contain a database of template bone models that are used by a variety of different planning computers in a variety of different locations.

The entire CAOS process may occur as part of a distributed computer network. For example, the initial X-rays of the patient may be taken at a local hospital and then sent electronically to a modeling computer in a central location. The operator of the modeling computer may search a local or remote database of template bone models to determine which model most closely resembles the patient's bone. Thereafter, the “morphing” of the model may take place on this same modeling computer, in this same location, or on a separate morphing computer at a different location.

Once the “morphed” patient bone model is generated, the model is preferably sent to a planning computer which aids in the determination of the pre-surgical plan. This planning computer may be located back at the original local hospital, or it may exist in some other location. The planning computer may be operated by a local operator, or the planning computer may be run by a remote expert. For example, the operators at the central location may send a patient's medical history, X-rays, 3D template bone model, and other information to a remotely located orthopedic surgeon or other expert. This expert may use that information and his or her skill to generate the plan on a local planning computer, or the expert may send plan suggestions back to a planning computer at the central location. The particular expert chosen to assist i developing the plan may be based on that expert's area of expertise.

After the generated (or amended) pre-surgical plan has bees accepted by the surgeon, the operation is performed. During the osteotomy surgery, fluoroscopic images of the marker positions are taker and then sent electronically to the planning computer (either in the same hospital or a remote location). The surgical plan can be updated, and the results of the updated surgical plan can be sent to the local hospital whet the osteotomy is performed.

Because of the segmented approach to the present orthopedic surgery method, the possibilities of patient and computer locations are virtually endless. These methods provide for “remote expertise” wherein CAOS experts can oversee and run the planning computer from a central location and a plurality of surgeons from different hospitals can electronically communicate with the CAOS experts. This method may include vastly reduced costs compared to present methods, and many hospitals and offices that can not afford IR tracking equipment will now be able to perform osteotomy procedures.

The above examples focused on an open wedge osteotomy ac an example of an orthopedic surgery performed using the present invention. However, this invention can be used for many different types of orthopedic surgery, as well as many other types of surgical and nonsurgical applications where intra-operative feedback may be helpful. For example, the present invention could also be used for closing wedge, distraction, dome, derotational, step-cut, and other types of orthopedic surgery. With these surgeries, the basic framework of the invention remains constant, but the exact plan and surgical tools used to implement the invention may be altered.

The present invention may also be used for a total joint replacement, such as a hip or knee replacement. For example, if the hinge surface of a patient's knee is worn out, the surgeon may cut the lower portion of the femur and the upper portion of the tibia and insert a new knee joint into the patient's leg. To achieve surgical success, the surgeon needs to align the new knee joint with the existing bone structure of the patient. Traditionally, a series of jigs and/or alignment rods have been used. Using the multifunctional markers of the present invention, the surgeon may be able to more accurately align the new joint using a less invasive procedure than conventional methods.

The present invention may be used in cases of multiple trauma with long bone fractures. To realign the bone and minimize bloc loss, the trauma surgeon uses an external fixator to quickly stabilize the patient. Thereafter, the surgeon may take a fluoroscopic or other image the fractures and apply the present system to obtain an exact realignment of the fractured bone.

The present invention may also be used for oncology-related applications, such as removing a bone tumor from a patient. Generally, surgeon performing a bone tumor removal seeks to remove only the tumorous portions of the bone while leaving the healthy tissue in tact. Because visual clues are not always available to the surgeon, the present invention may be used to develop a surgical plan and place markers around the tumor sight. An updated image of the marker position may b used to easily determine which parts of the bone are tumorous and need be removed. Also, after the surgery, the markers may be used to make sure that the complete tumor was removed. Use of the present invention is less expensive and time consuming than the conventional MRI/CAT-based methods.

The present invention may also be used to ease the performance of complicated surgeries. For example, spine surgery may be difficult because it involves a 3D surgery around the spine in an area of the body where there may be a small margin for error. The multifunctional markers and fixation devices may allow the surgery to be performed more precisely, and in a reduced amount of time.

The present invention may also be used to perform intramedullary procedures on a patient's bone. In such a procedure, a rod is inserted inside the hollow of a bone down its long axis. Near at least one end of the rod, there is an elliptical hole that accepts a screw to prevent the rod from rotating or twisting within the bone. In convention-, methods, it is often difficult to accurately locate the elliptical hole for the set screw. Using the multifunctional markers and fixation devices of the present invention, localization would be more easily accomplished.

For example, the updated marker position may be used to provide settings as part of a surgical plan for a device that allows the insertion of the set screw. Rather than searching within the patient to find the elliptical hole, the surgeon can set the fixation device and insert the screw with confidence that the hole will be beneath the device.

The present invention may be used in a similar manner to the above methods for performing localization and surgical procedures or. bone lesions, any soft tissues, and/or maxilo-facial surgery. In general, the embodiments and features of the present invention may be specifically suited to aiding in the performance of many or all bone and soft tissue procedures.

The above specification describes several different embodiments and features of a device and method for performing orthopedic surgery. Various parts, selections, and/or alternatives from various embodiments may preferably be interchanged with other parts to different embodiments. Although the invention has been described above in terms of particular embodiments, one of ordinary skill in the art, in light of the teachings herein, can generate additional embodiments and modifications without departing from the spirit of, or exceeding the scope of, the claimed invention. Accordingly, it is to be understood that the drawings and the descriptions herein are proffered only by way of example only to facilitate comprehension of the invention and should not be construed to limit the scope thereof.

While several embodiments of the invention have been described, it should be apparent, however, that various modifications, alterations and adaptations to those embodiments may occur to persons skilled in the art with the attainment of some or all of the advantages of the present invention. It is therefore intended to cover all such modifications, alterations and adaptations without departing from the scope and spirit of the present invention as defined by the appended claims.

1. A method of generating a three-dimensional (3D) model of a bone, the method comprising:
determining one or more contours of the bone based on one or more two dimensional (2D) images of the bone, and
based on the one or more contours of the bone, modifying a 3D template model of the bone to generate a 3D model of the bone.

2. The method of claim 1, wherein the 2D images are orthogonal to each other.

3. The method of claim 1, wherein the 2D images include digitized images.

4. The method of claim 1, wherein the 2D images include one or more X-ray images.

5. The method of claim 1, wherein determining one or more contours includes:
determining a 2D fiducial geometry of the bone based on the one or more images of the bone.

6. The method of claim 1, further comprising:
providing a database of 3D template models of bones, and
based on the one or more contours of the bone, selecting a 3D template model of the bone from the database.

7. The method of claim 1, wherein modifying includes:
modifying one or more of a size and a position of the 3D template model.

8. The method of claim 1, wherein modifying includes:
deforming the 3D template model.

9. The method of claim 8, wherein deforming includes:
based on the 3D template model, determining the 2D fiducial geometry of the bone, and
based on the 2D fiducial geometry, deforming the 3D template model to generate a 3D model having 2D projections that are similar to the 2D images.

10. The method of claim 9, wherein deforming includes:
based on the 3D template model, generating a 3D lattice, and
deforming the 3D lattice to generate 2D projections that are similar to the 2D images.

11. The method of claim 10, wherein deforming the 3D lattice includes:
computing one or more free form deformation parameters for the 3D lattice, and
iteratively determining values of the FFD parameters for generating 2D projections that are similar to the 2D images.

12. The method of claim 11, wherein the 3D lattice includes one or more parallelpipeds.

13. A method of generating a three dimensional (3D) model of a bone, the method comprising:
based on one or more two dimensional (2D) images of the bone, identifying a 3D template model of the bone, and
modifying the 3D template model of the bone to generate a 3D model of the bone having 2D projections that are similar to the 2D images.

14. The method of claim 13, wherein determining one or more contours includes:
determining a 2D fiducial geometry of the bone based on the one or more images of the bone.

15. The method of claim 13, further comprising:
providing a database of 3D template models of bones, and
based on the one or more contours of the bone, selecting a 3D template model of the bone from the database.

16. The method of claim 13, wherein modifying includes:
modifying one or more of a size and a position of the 3D template model.

17. The method of claim 13, wherein modifying includes:
deforming the 3D template model.

18. The method of claim 17, wherein deforming includes:
based on the 3D template model, determining the 2D fiducial geometry of the bone, and
based on the 2D fiducial geometry, deforming the 3D template model to generate a 3D model having 2D projections that are similar to the 2D images.

19. The method of claim 18, wherein deforming includes:
based on the 3D template model, generating a 3D lattice, and
deforming the 3D lattice to generate 2D projections that are similar to the 2D images.

20. The method of claim 19, wherein deforming the 3D lattice includes:
computing one or more free form deformation parameters for the 3D lattice, and
iteratively determining values of the FFD parameters for generating 2D projections that are similar to the 2D images.
